(19) United States
(12) Patent Application Publication
(10) Pub. No.: US 2018/0340933 A1
(43) Pub. Date: Nov. 29, 2018

(54) Soybean Allergy Related Epitopes

(71) Applicant: Fraunhofer-Gesellschaft zur Foerderung der angewandten Forschung e.V., Muenchen (DE)

(72) Inventors: Karolin Kern, Halle (DE); Holger Spiegel, Aachen (DE); Heide Havenith, Aachen (DE); Michael Szardenings, Leipzig (DE)

(73) Assignee: Fraunhofer-Gesellschaft zur Foerderung der angewan Forschung e.V., Muenchen (DE)

(21) Appl. No.: 15/757,941

(22) PCT Filed: Sep. 8, 2016
(86) PCT No.: PCT/EP2016/071196

§ 371 Date: Mar. 6, 2018

(30) Foreign Application Priority Data
Sep. 8, 2015 (EP) 15184199.6

(51) Int. Cl. G01N 33/569; C07K 14/415

(52) U.S. Cl. CPC G01N 33/56961; G01N 2800/24; G01N 2333/415; C07K 14/415

(57) ABSTRACT

The invention relates to a compilation comprising at least five different peptides, each peptide comprising at least one sequence element corresponding to an epitope selected from the group consisting of SEQ ID NO.: 1-354, wherein at least five different epitopes are represented. The invention further relates to an in vitro method for determining a patient's immune status to soybean allergens, to a method for detecting at least one soybean allergen in a substance and to a method for determining the allergenicity of a soybean variety. Additionally, the invention relates to a kit comprising at least one composition containing a compound comprising at least five different sequence elements each corresponding to an epitope selected from the group consisting of SEQ ID NO.: 1-354, wherein at least five different epitopes are represented. Furthermore, the invention relates to the use of a peptide comprising a sequence element corresponding to an epitope for providing a molecule binding to a protein or peptide comprising the epitope.

### FIELD OF THE INVENTION

The invention relates to a compilation comprising at least five different peptides, each peptide comprising at least one sequence element corresponding to an epitope selected from the group consisting of SEQ ID NO.: 1-354, wherein at least five different epitopes are represented. The invention further relates to an in vitro method for determining a patient's immune status to soybean allergens, to a method for detecting at least one soybean allergen in a substance and to a method for determining the allergenicity of a soybean variety. Additionally, the invention relates to a kit comprising at least one composition containing a compound comprising at least five different sequence elements each corresponding to an epitope selected from the group consisting of SEQ ID NO.: 1-354, wherein at least five different epitopes are represented. Furthermore, the invention relates to the use of a peptide comprising a sequence element corresponding to an epitope for providing a molecule binding to a protein or peptide comprising the epitope. The invention further relates to a method for producing an immunoassay product and to a method for generating a variant of an allergenic soybean protein having a reduced allergenic potential.

### BACKGROUND OF THE INVENTION

Within the last decades, soybean seeds have become an important source of protein in many countries of the world. In developing countries, soybean is increasingly used to provide sufficient nutrition to the population, whereas in industrial countries it has become a major ingredient of processed food products. Moreover, soybean food products are specifically promoted for having health benefits and are believed to lower incidences of high plasma cholesterol, cancer, diabetes mellitus and obesity. Indeed, soybean products provide all essential amino acids and are rich in vitamins and minerals, making them an interesting source for protein rich nutrition. Despite these advantages, soybean is one of the most allergenic foods. Soybean allergies usually arise spontaneously during childhood with symptoms such as atopic dermatitis, enterocolitis and other IgE-mediated multisystem reactions (Masilamani et al., 2012).

Moreover, they often occur together with allergies to other food products, in particular peanut and milk protein (Masilamani et al., 2012). Thus, affected patients not only have to cope with the risk of allergic reactions for almost their entire life but need to be particularly careful about their diet in general.

Although the reaction threshold of soybean protein (400 mg) is rather high compared to peanut (0.1 mg), most sensitive persons need to avoid soybean proteins completely to prevent allergic reaction (Masilamani et al., 2012). Unfortunately, obviating soybean protein containing food products is increasingly difficult, since soybean protein became an ubiquitous ingredient of vegetable as well as meat containing foods. In particular processed or partially processed food products usually contain at least traces of at least some soybean proteins. To increase security for persons suffering from allergies, governments, in particular within the European Union and the United States of America, implement increasing requirements regarding the identification of food ingredients. To meet these requirements, food producing companies need comprehensive and reliable methods for analyzing the content of their food products.

Additionally, patients need to know about the primary cause and extent of their allergic reaction to be able to benefit from the increasing information provided about food product ingredients.

Therefore, an unmet need exists for improved products and methods to determine the sensitivity of patients to soybean protein as well as to identify the presence of soybean proteins and their allergenic potential in food and other products.

### SUMMARY OF THE INVENTION

In a first aspect, the invention relates to a compilation comprising at least five different peptides, each peptide comprising at least one sequence element corresponding to an epitope selected from the group consisting of SEQ ID NO.: 1-354, wherein at least five different epitopes are represented.

In a further aspect, the invention relates to an in vitro method for determining a patient's immune status to soybean allergens, comprising the steps providing at least five different peptides, each peptide comprising at least one sequence element corresponding to an epitope selected from the group consisting of SEQ ID NO.: 1-354, wherein at least five different epitopes are represented, contacting each peptide with a sample comprising antibodies derived from the patient, and detecting an interaction of each peptide with the sample.

In a further aspect, the invention relates to a kit comprising at least one composition containing a compound comprising at least five different sequence elements each corresponding to an epitope selected from the group consisting of SEQ ID NO.: 1-354, wherein at least five different epitopes are represented.

In a further aspect, the invention relates to a method for detecting at least one soybean allergen in a substance, comprising the steps providing at least two different peptides, each peptide comprising at least one sequence element corresponding to an epitope selected from the group consisting of SEQ ID NO.: 1-354, raising at least one antibody against each peptide, contacting the substance with the antibodies, and determining binding of the antibodies to the substance.

In a further aspect, the invention relates to a method for producing an immunoassay product, comprising the steps providing at least two different peptides, each peptide comprising at least one sequence element corresponding to an epitope selected from the group consisting of SEQ ID NO.: 1-354, raising at least one antibody against each peptide, and compiling the antibodies to provide the immunoassay product.

In a further aspect, the invention relates to a method for determining the allergenicity of a soybean variety by detecting the presence of at least two epitopes in a sample of the variety, wherein the epitopes are selected from the group consisting of SEQ ID NO.: 1-354.

In a further aspect, the invention relates to the use of a peptide comprising a sequence element corresponding to an epitope selected from the group consisting of SEQ ID NO.: 1-354 for providing a molecule binding to a protein or peptide comprising the epitope.

In a further aspect, the invention relates to a method for generating a variant of an allergenic soybean protein having a reduced allergenic potential, comprising the steps providing an amino acid sequence of at least one epitope selected from the group consisting of SEQ ID NO.: 1-354, altering the amino acid sequence of the at least one epitope as to eliminating the structure of the epitope, and generating a protein or peptide comprising the altered amino acid sequence.

### BRIEF DESCRIPTION OF THE DRAWINGS

FIG. 1 depicts the procedure of identifying epitopes responsible for immunologic reactions to soybean protein.

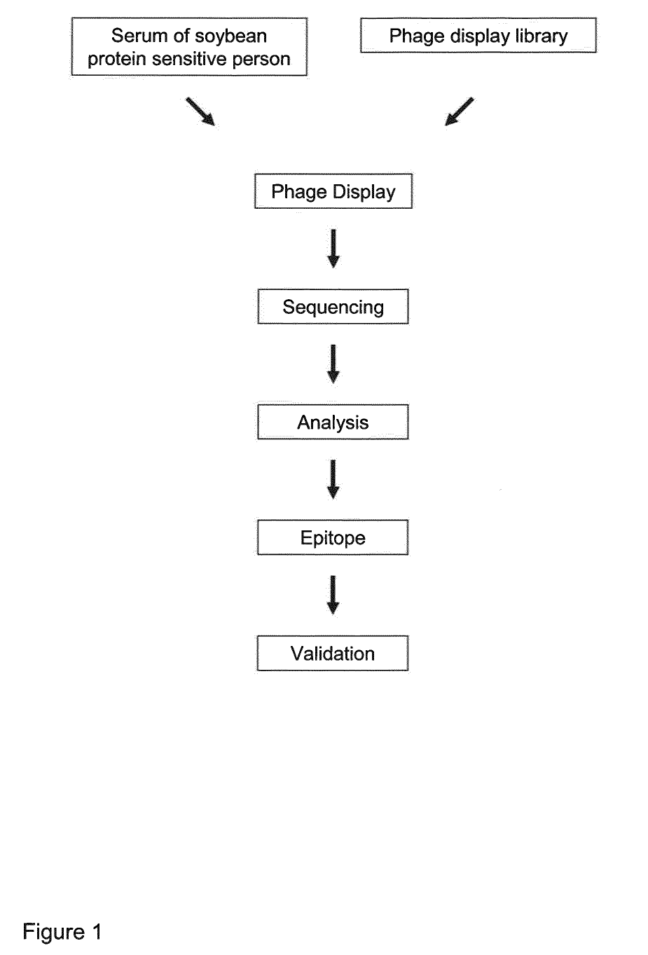

FIG. 2 depicts the phage display procedure used for identifying epitopes.

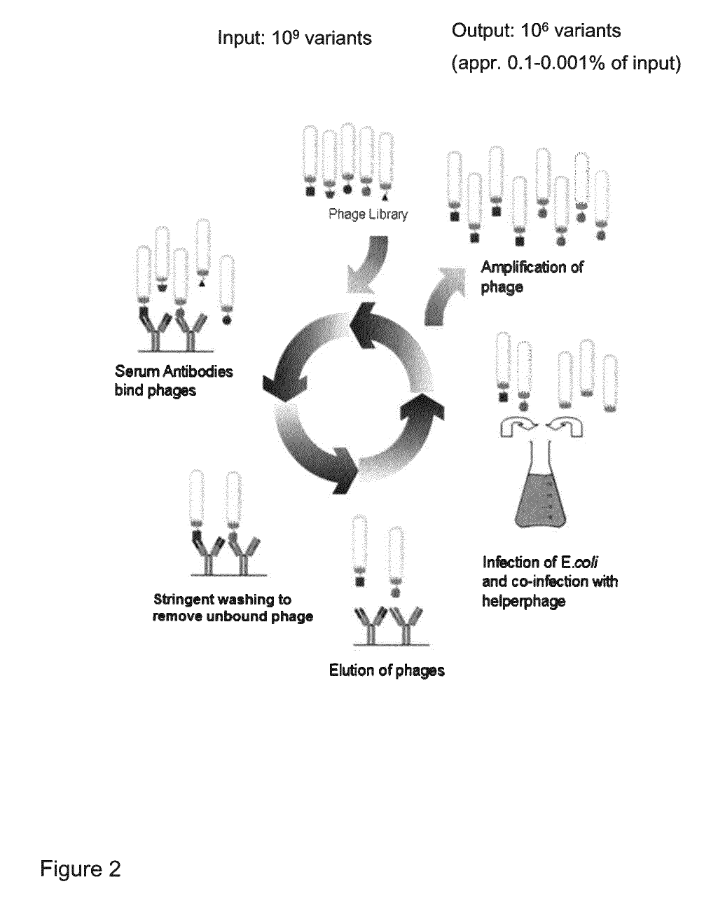

FIG. 3 depicts the size of allergy relevant sequences of soybean proteins identified by various techniques in relation to the size of an entire soybean protein. Whereas the free epitope data base (www.IEDB.org) and arrays of synthetic peptides for screening protein sequences provide sequences of about 10 to 20 and even more amino acids, specific core sequences for anti-Allergen Antibody binding peptides of usually 4 to 6 amino acids were identified by phage display.

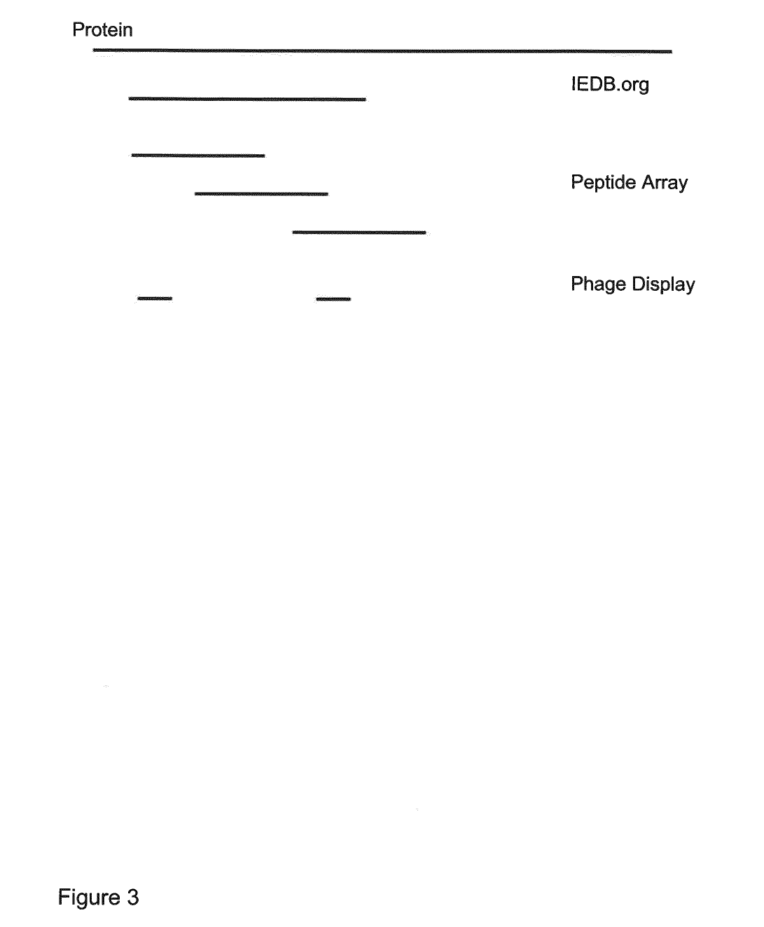

FIG. 4 shows the identification of an epitope. “Seq” refers to the original sequence tested in PEPper-PRINT Array. All sequences identified from Serum 21 (only a subset shown) share the common motif VFASQV (SEQ ID NO.: 94). The motifs variability is shown by different sequences bound by serum 21.

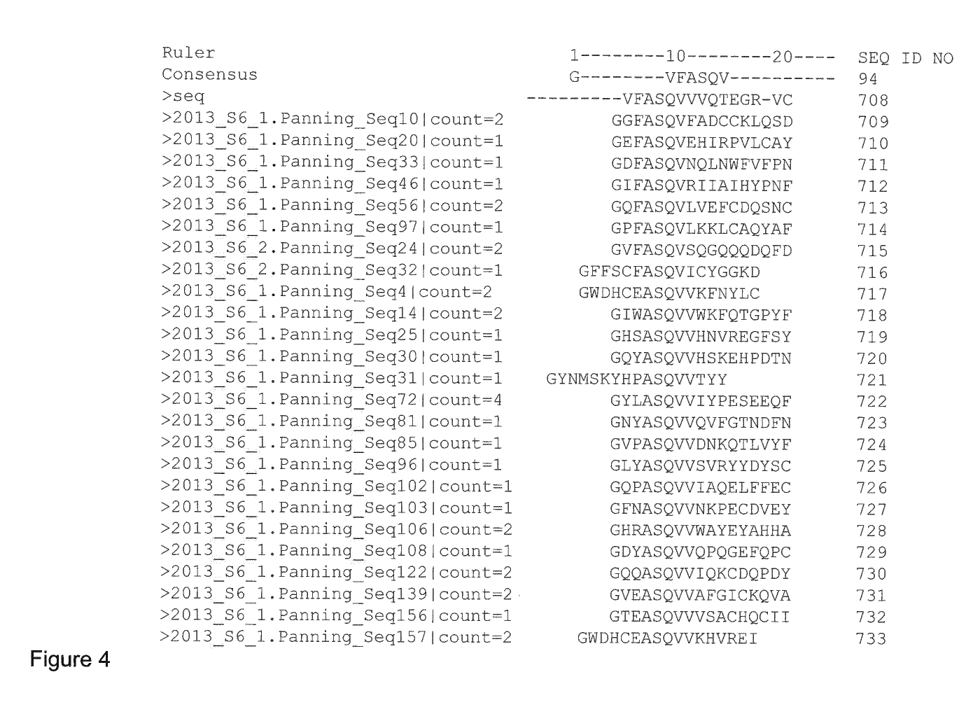

### DETAILED DESCRIPTION OF THE INVENTION

In a first aspect, the invention relates to a compilation comprising at least five different peptides, each peptide comprising at least one sequence element corresponding to an epitope selected from the group consisting of SEQ ID NO.: 1-354, wherein at least five different epitopes are represented.

Several proteins of soybean seeds have been found to trigger immunoglobulin E (IgE) mediated allergic responses in sensitive persons. Strong immunodominant allergic proteins, such as P34 thiol protease (Gly m Bd 30K), are known to induce allergic reactions, whereas other soybean proteins, although no potent inducers of a primary allergic response, can nevertheless trigger effector responses due to their binding by IgE. Interestingly, affected persons show varying sensitivity to different soybean proteins. For example, only 65% of soybean sensitive individuals carry IgE recognizing P34 thiol protease (Gly m Bd 30K), which is considered one of the strongest allergenic soybean proteins (Yang et al., 2011). Finally, as antibodies usually do not recognize an entire protein but interact with distinct structures of limited numbers of amino acids within a molecule, sensitivity between patients varies distinctly due to their genetic background and different results of antibody maturation. To identify many specific epitopes, i.e. amino acid sequences of soybean proteins, recognized by antibodies of sensitive patients, with high resolution down to the individual amino acids involved, the inventors applied a novel approach using a specific random phage library (EP 14 166 662.8). This phage library is generated using triplet codons with specific limitations, such that most amino acids are regularly represented throughout the entire library. In consequence, the library covers an unusually broad range of sequence variability and allows particular reliable statistical analysis. The library was contacted with serum samples derived from a biobank of 50 different soybean protein sensitive patients. After phage selection, the amino acid sequences that were bound by the patients' antibodies (anti-[allergen antibody] amino acid sequence) were identified by next generation sequencing and the results statistically analyzed. To confirm the allergy relevance of the identified amino acid sequences, their reactivity with patient IgE was determined by peptide arrays. Thereby, the inventors identified more than 300 potentially allergenic epitopes from different soybean proteins. The identified epitopes could be assigned to a total of 23 soybean proteins (Table 1) including P34 thiol protease (Gly m Bd 30K), β-conglycinin (Gly m Bd60 K) and Gly m Bd 28K, which are considered major soybean allergenic proteins (Yang et al., 2011).

TABLE 1
Individual Epitopes

| | | | SEQ ID |
|---|---|---|---|
| Soybean allergen (ID) | Epitop NO | Epitop sequence | NO. |
| beta conglycinin alpha chain (X17698) | 6.04.001 | SNRFE | 1 |
| | 6.04.002 | FKNQYG | 2 |
| | 6.04.003 | FNSKPN | 3 |
| | 6.04.004 | VNPDNN | 4 |
| | 6.04.005 | LAIPV | 5 |
| | 6.04.006 | NXPGRFES | 6 |
| | 6.04.007 | EASYD | 7 |
| | 6.04.008 | EQQQGE | 8 |
| | 6.04.009 | QESVI | 9 |
| | 6.04.010 | KSSSRK | 10 |
| | 6.04.011 | PEKNPQ | 11 |
| | 6.04.012 | FVIPAG | 12 |
| | 6.04.013 | AFPGS | 13 |
| | 6.04.014 | CLQSCNS | 14 |
| | 6.04.015 | NSERD | 15 |
| | 6.04.016 | ERDSYR | 16 |
| | 6.04.017 | RNQACHA | 17 |
| | 6.04.018 | QACHAR | 18 |
| | 6.04.019 | KEECEEG | 19 |
| | 6.04.020 | EIPRPR | 20 |
| | 6.04.021 | KEECEE | 21 |
| | 6.04.022 | RPQHPEREP | 22 |
| | 6.04.023 | REPQQPGE | 23 |
| | 6.04.024 | HPEREP | 24 |
| | 6.04.025 | QQPGEKE | 25 |
| | 6.04.026 | EDEDEQPRP | 26 |
| | 6.04.027 | IPFPRPQP | 27 |
| | 6.04.028 | PRQEEE | 28 |
| | 6.04.029 | EEHEQREEQ | 29 |
| | 6.04.030 | GEKGSEE | 30 |
| | 6.04.031 | RNEEEDE | 31 |
| | 6.04.032 | EDEEQQ | 32 |
| | 6.04.033 | EEQQRES | 33 |
| | 6.04.034 | RESEES | 34 |
| | 6.04.035 | VLFSREEG | 35 |
| | 6.04.036 | EEGQQQG | 36 |
| | 6.04.037 | LRSRDPIY | 37 |
| | 6.04.038 | LSIVD | 38 |
| | 6.04.039 | LKEQQQE | 39 |
| | 6.04.040 | EQQQE | 40 |
| | 6.04.041 | EEQPLE | 41 |
| | 6.04.042 | NQRESYFV | 42 |
| | 6.04.043 | FVDAQP | 43 |
| beta conglycinin alpha′ chain (M13759.1) | 7.03.001 | GVVFL | 44 |
| | 7.03.002 | RFQTL | 45 |
| | 7.03.003 | PQLRD | 46 |
| | 7.03.005 | QPHQK | 47 |
| | 7.03.007 | DRDSY | 48 |
| | 7.03.008 | LRVPA | 49 |
| | 7.03.009 | KVEEEE | 50 |
| | 7.03.010 | PERERQ | 51 |
| | 7.03.011 | QQHGEK | 52 |
| | 7.03.012 | EDEGEQP | 53 |
| | 7.03.013 | EGEQPRPF | 54 |
| | 7.03.014 | FPRPRQP | 55 |
| | 7.03.015 | QPHQEE | 56 |
| | 7.03.016 | EQKEEH | 57 |
| | 7.03.017 | KEEHE | 58 |
| | 7.03.018 | SEEEQDERE | 59 |
| | 7.03.019 | HKQEKH | 60 |
| | 7.03.020 | ESEEE | 61 |
| | 7.03.021 | EEEDQDE | 62 |
| | 7.03.022 | EEDQDEDEEQ | 63 |
| | 7.03.023 | ESQESEGSE | 64 |
| | 7.03.024 | ESQREP | 65 |
| | 7.03.025 | NKRSQQLQ | 66 |
| | 7.03.026 | LPHHAD | 67 |
| | 7.03.027 | GREEGQ | 68 |
| | 7.03.028 | VLVINE | 69 |
| | 7.03.029 | IELVGI | 70 |
| | 7.03.030 | QDIFVIP | 71 |
| | 7.03.031 | DAQPQQ | 72 |
| | 7.03.032 | LEVRKYR | 73 |
| | 7.03.033 | ESYFVD | 74 |
| | 7.03.034 | FVDAQP | 75 |
| beta conglycinin beta chain (S44893) | 8.04.001 | FLASV | 76 |
| | 8.04.002 | LSGRAI | 77 |
| | 8.04.005 | HQNLK | 78 |
| | 8.04.006 | KPGRYD | 79 |
| | 8.04.007 | ELSKEQ | 80 |
| | 8.04.010 | IERQVQ | 81 |
| | 8.04.011 | PQLENL | 82 |
| | 8.04.012 | DSYNLH | 83 |
| | 8.04.013 | PGDAQ | 84 |
| | 8.04.014 | SHNILE | 85 |
| | 8.04.015 | SFHSEFEE | 86 |
| | 8.04.016 | EEINRV | 87 |
| | 8.04.017 | RVLFGE | 88 |
| | 8.04.018 | QRQQE | 89 |
| | 8.04.019 | QEGVIV | 90 |
| | 8.04.020 | ILVINE | 91 |
| | 08.04.2008 | PHFNSK | 92 |
| | 08.04.2009 | EEEPLE | 93 |
| Defensin (Z13956.1) | 10.00.01 | VFASQV | 94 |
| | 10.00.02 | VVVQTE | 95 |
| | 10.00.03 | TEGRVC | 96 |
| | 10.00.04 | RVCESQS | 97 |
| | 10.00.05 | QSHGF | 98 |
| | 10.00.06 | SQSHGFH | 99 |
| | 10.00.07 | HGLCNRDHN | 100 |
| | 10.00.08 | RDHNCALVC | 101 |
| | 10.00.09 | LVCRNE | 102 |
| | 10.00.10 | SRRCF | 103 |
| Gly m BD 28K (AB046874.2) | 11.00.01 | QEEDEE | 104 |
| Glycinin G1 (AB113349.1) | 12.00.01 | QCAGVA | 105 |
| | 12.00.02 | PRGSQS | 106 |
| | 12.00.02 | VPHYNLNA | 107 |
| | 12.00.03 | LIQVVN | 108 |
| | 12.00.04 | AARSQ | 109 |
| | 12.00.05 | DNFEY | 110 |
| | 12.00.06 | SREQPQ | 111 |
| | 12.00.07 | CCFAFS | 112 |
| | 12.00.08 | PQQNEC | 113 |
| | 12.00.09 | GPQEIYIQQ | 114 |
| | 12.00.10 | RPSYTN | 115 |
| | 12.00.11 | CPSTFEE | 116 |
| | 12.00.12 | IYPGCPS | 117 |
| | 12.00.13 | PQQPQQR | 118 |
| | 12.00.14 | QRGQS | 119 |
| | 12.00.15 | QSSRPQ | 120 |
| | 12.00.16 | PQDRHQ | 121 |
| | 12.00.17 | QKIYNF | 122 |
| | 12.00.18 | REGDL | 123 |
| | 12.00.19 | VPTGVAW | 124 |
| | 12.00.20 | NQLDQ | 125 |
| | 12.00.21 | QEQEF | 126 |
| | 12.00.22 | KYQQEQ | 127 |
| | 12.00.23 | HQSQK | 128 |
| | 12.00.24 | KHQQEEEN | 129 |
| | 12.00.25 | KNLQGE | 130 |
| | 12.00.26 | TDEQQQ | 131 |
| | 12.00.27 | QQRPQE | 132 |
| | 12.00.28 | EKPQCK | 133 |
| | 12.00.29 | DKHCQR | 134 |
| | 12.00.30 | FVPHY | 135 |
| | 12.00.31 | KFLVPPQE | 136 |
| | 12.00.32 | PQESQK | 137 |
| Glycinin G2 (D00216.1) | 13.00.01 | CPSTYN | 138 |
| | 13.00.02 | QGGSQS | 139 |
| | 13.00.03 | SGAIV | 140 |
| | 13.00.03 | GGLRVT | 141 |
| | 13.00.04 | RLRQN | 142 |
| | 13.00.05 | LKLSAQ | 143 |
| | 13.00.06 | VAAKSQ | 144 |
| | 13.00.07 | PSIGNL | 145 |
| | 13.00.08 | FSFLVPP | 146 |
| | 13.00.09 | REQAQQN | 147 |
| | 13.00.10 | QKLNA | 148 |
| | 13.00.11 | RIESEG | 149 |
| | 13.00.12 | TYQEPQ | 150 |
| | 13.00.13 | PQESQ | 151 |
| | 13.00.14 | ESQQRG | 152 |
| | 13.00.15 | GRSQR | 153 |
| | 13.00.16 | QDRHQK | 154 |
| | 13.00.17 | VHRFRE | 155 |
| | 13.00.18 | EEENEG | 156 |
| | 13.00.19 | QIVRNL | 157 |
| | 13.00.20 | RKPQQE | 158 |
| | 13.00.21 | EEEQPQ | 159 |
| | 13.00.22 | DIYNPQ | 160 |
| | 13.00.23 | ERVFD | 161 |
| | 13.00.24 | ELQEG | 162 |
| Glycinin G3 (X15123.1) | 14.00.01 | FREQPQQN | 163 |
| | 14.00.02 | QPQKQQ | 164 |
| | 14.00.03 | RQIVRK | 165 |
| | 14.00.04 | RRQQAR | 166 |
| | 14.00.05 | ECQIQR | 167 |
| | 14.00.06 | IQQGSGI | 168 |
| | 14.00.07 | QQKGQS | 169 |
| | 14.00.08 | IYHFRE | 170 |
| | 14.00.09 | AVPTGF | 171 |
| | 14.00.10 | NSFQN | 172 |
| | 14.00.11 | PTEEQ | 173 |
| | 14.00.12 | QQQRPE | 174 |
| | 14.00.13 | PDCDEK | 175 |
| | 14.00.14 | DKHCQS | 176 |
| | 14.00.15 | CQSQSR | 177 |
| | 14.00.16 | QSQSRN | 178 |
| | 14.00.17 | HNIGQT | 179 |
| Glycinin G4 (X05652) | 15.03.2001 | QGKGA | 180 |
| | 15.03.2002 | VLSGFS | 181 |
| | 15.03.2003 | LNECQL | 182 |
| | 15.03.2004 | PDHRVE | 183 |
| | 15.03.2005 | RNGLHS | 184 |
| | 15.03.2006 | YSPYPR | 185 |
| | 15.03.2007 | VAIPGC | 186 |
| | 15.03.2008 | ETFEEP | 187 |
| | 15.03.2009 | QQLQDS | 188 |
| | 15.03.2010 | IRHFNE | 189 |
| | 15.03.2011 | DIEYPET | 190 |
| | 15.03.2012 | KQGQHQ | 191 |
| | 15.03.2013 | DIAEKL | 192 |
| | 15.03.2014 | KLESPD | 193 |
| | 15.03.2015 | SVISPK | 194 |
| | 15.03.2016 | QQDED | 195 |
| | 15.03.2017 | KREQD | 196 |
| | 15.03.2018 | HPPRRP | 197 |
| | 15.03.2019 | REQDED | 198 |
| | 15.03.2020 | GQDEDE | 199 |
| | 15.03.2021 | TQPRRP | 200 |
| | 15.03.2022 | PRQEEP | 201 |
| | 15.03.2023 | EEPRER | 202 |
| Glycinin G5 (M10962.1) | 16.02.2001 | NSQHPE | 203 |
| | 16.02.2002 | HPELQC | 204 |
| | 16.02.2003 | QCAGVT | 205 |
| | 16.02.2004 | TVSKR | 206 |
| | 16.02.2005 | KGAIGF | 207 |
| | 16.02.2006 | GSRSQ | 208 |
| | 16.02.2007 | VTVEG | 209 |
| | 16.02.2008 | RTPSYPP | 210 |
| | 16.02.2009 | VEENICT | 211 |
| | 16.02.2010 | PSRADF | 212 |
| | 16.02.2011 | NCQGN | 213 |
| | 16.02.2012 | RRGQLL | 214 |
| | 16.02.2013 | PAVAE | 215 |
| | 16.02.2014 | IPSEVL | 216 |
| | 16.02.2015 | NSYNL | 217 |
| | 16.02.2016 | CQLNN | 218 |
| | 16.02.2017 | RVESEG | 219 |
| | 16.02.2018 | YLPYPQ | 220 |
| | 16.02.2019 | SHLPSY | 221 |
| | 16.02.2020 | IGFAFP | 222 |
| | 16.02.2021 | FPGCPE | 223 |
| | 16.02.2022 | VIPLGV | 224 |
| | 16.02.2023 | DEPVVA | 225 |
| | 16.02.2024 | RVFYL | 226 |
| | 16.02.2025 | GNPDIE | 227 |
| | 16.02.2026 | IEHPET | 228 |
| | 16.02.2027 | QGQHR | 229 |
| | 16.02.2028 | HRQQE | 230 |
| | 16.02.2029 | QQEEEG | 231 |
| | 16.02.1930 | DTAEK | 232 |
| | 16.02.1931 | EGGLS | 233 |
| | 16.02.1932 | KWQEQE | 234 |
| | 16.02.1933 | QEQEDE | 235 |
| | 16.02.1934 | DEEYGR | 236 |
| | 16.02.1935 | GKHEDD | 237 |
| | 16.02.1936 | EEEDQP | 238 |
| | 16.02.1937 | PRPDHP | 239 |
| | 16.02.1938 | HPPQRP | 240 |
| | 16.02.1939 | QRPSRP | 241 |
| | 16.02.1940 | PEQQEP | 242 |
| | 16.02.1941 | QQEPRG | 243 |
| | 16.02.1942 | LRRGQ | 244 |
| | 16.02.1943 | GNAVFD | 245 |
| | 16.02.1944 | THHNAV | 246 |
| | 16.02.1945 | SNSYNL | 247 |
| | 16.02.1946 | LGQSQV | 248 |
| | 16.02.1947 | PLVNP | 249 |
| | 16.02.1948 | QGNSG | 250 |
| Hydrophobic Seed Protein (A29385) | 18.00.01 | LGGSL | 251 |
| | 18.00.02 | DDCCAL | 252 |
| | 18.00.03 | NSCGRS | 253 |
| Kunitz trypsin inhibitor (S45092.1) | 19.13.01 | GNERC | 254 |
| | 19.13.02 | RAAPTG | 255 |
| | 19.13.03 | LCVGIP | 256 |
| | 19.13.04 | TEWSV | 257 |
| p34 thiol protease (J05560.1) | 23.01.2001 | VITQVK | 258 |
| | 23.01.2002 | KYQGGC | 259 |
| Profilin (AJ223981.1) | 26.00.01 | KPEEI | 260 |
| | 26.00.02 | FPQFKP | 261 |
| | 26.00.03 | EPGSL | 262 |
| | 26.00.04 | QGEPGAV | 263 |
| | 26.00.05 | ERLGDY | 264 |
| | 26.00.06 | AYVDDH | 265 |
| | 26.00.07 | NHLTHAAI | 266 |
| | 26.00.08 | IIGQDG | 267 |
| | 26.00.09 | GSVWLQ | 268 |
| | 26.00.10 | TPGQC | 269 |
| | 26.00.11 | IYDEP | 270 |
| Profilin (AJ223982.1) | 26.01.2001 | CDIEGN | 271 |
| | 26.01.2002 | GSVWAQ | 272 |
| | 26.01.2003 | VVERP | 273 |
| | 26.01.2004 | IDQGY | 274 |
| PR10 (X60043.1) | 25.00.01 | SIDEA | 275 |
| | 25.00.02 | NLGYSY | 276 |
| | 25.00.03 | VENVEGN | 277 |
| | 25.00.04 | EDEINS | 278 |
| | 25.00.05 | NSPVAP | 279 |
| | 25.00.06 | PDTAEK | 280 |
| | 25.00.07 | EKITF | 281 |
| | 25.00.08 | GSAGKLT | 282 |
| | 25.00.09 | ETKGD | 283 |
| 18 kD Seed Maturation Protein | 2.00.001 | IGASA | 284 |
| (AJ574791.1) | 2.00.002 | ATVQEKAER | 285 |
| | 2.00.003 | ARDPVQ | 286 |
| | 2.00.004 | ELATQK | 287 |
| | 2.00.005 | QHNTA | 288 |
| | 2.00.006 | GHGHHT | 289 |
| | 2.00.007 | GEYGQP | 290 |
| | 2.00.008 | HQTSA | 291 |
| | 2.00.009 | HGTGQPTGH | 292 |
| | 2.00.010 | HVTEGV | 293 |
| | 2.00.011 | VGSHPIG | 294 |
| 2s Albumin (AAB71140.1) | 1.00.001 | LFCIAHTCS | 295 |
| | 1.00.002 | SASKWQH | 296 |
| | 1.00.004 | QQDSCR | 297 |
| | 1.00.005 | SCRKQL | 298 |
| | 1.00.006 | KQLQGVN | 299 |
| | 1.00.007 | NLTPCEK | 300 |
| | 1.00.009 | QGRGD | 301 |
| | 1.00.011 | EDEEEEG | 302 |
| | 1.00.012 | QKCCT | 303 |
| | 1.00.013 | TEMSEL | 304 |
| | 1.00.015 | CKALQK | 305 |
| | 1.00.016 | NQSEELEEK | 306 |
| | 1.00.017 | MCRFGP | 307 |
| | 1.00.018 | IQCDLS | 308 |
| 2s Albumin (BT091363.1) | 1.01.002 | CASKWQQH | 309 |
| | 1.01.003 | QHQQES | 310 |
| | 1.01.005 | ESCREQL | 311 |
| | 1.01.007 | NPCEHI | 312 |
| | 1.01.008 | EKIQAG | 313 |
| | 1.01.009 | DGSDEDH | 314 |
| | 1.01.010 | EGKEE | 315 |
| | 1.01.011 | KEEEEE | 316 |
| | 1.01.013 | SEMSE | 317 |
| | 1.01.014 | SPICQCK | 318 |
| | 1.01.016 | NQSEQLEGKE | 319 |
| | 1.01.017 | AIRCRL | 320 |
| | 1.01.018 | IGCDL | 321 |
| Albumin 1 (AJ574791.1) | 3.00.001 | NGACSPFE | 322 |
| | 3.00.002 | PPCRSRD | 323 |
| | 3.00.003 | RDCRC | 324 |
| | 3.00.004 | RCVPIGL | 325 |
| | 3.00.005 | AGFCIH | 326 |
| | 3.00.006 | GLSSVA | 327 |
| | 3.00.008 | HPNLCQSD | 328 |
| | 3.00.009 | QSDDEC | 329 |
| | 3.00.010 | GNFCARYP | 330 |
| Albumin 1 (AJ223037.1) | 3.01.002 | PPCRSS | 331 |
| | 3.01.007 | VDEHP | 332 |
| | 3.01.011 | SDSEA | 333 |
| Albumin 1 (AJ223037.1) | 3.03.001 | EGTSSAKLT | 334 |
| | 3.03.002 | RCVPIA | 335 |
| Lectin (K00821.1) | 21.05.2001 | LAPIDT | 336 |
| | 21.05.2002 | TKPQT | 337 |
| | 21.05.2003 | KPQTHA | 338 |
| | 21.05.2004 | DPPNPHI | 339 |
| | 21.05.2005 | DTFRN | 340 |
| | 21.05.2006 | NLPHAS | 341 |
| Seed biotinylated protein (U59626.1) | 27.01.2001 | EIHVEK | 342 |
| | 27.01.2002 | KHRVPK | 343 |
| | 27.01.2003 | DHAGKA | 344 |
| | 27.01.2004 | KESQRE | 345 |
| | 27.01.2005 | ANVVG | 346 |
| | 27.01.2006 | VAEKGR | 347 |
| | 27.01.2007 | GRETE | 348 |
| | 27.01.2008 | AHVVEG | 349 |
| | 27.01.2009 | EYTAK | 350 |
| | 27.01.2010 | EAQRELE | 351 |
| | 27.01.2011 | QPQEAE | 352 |
| | 27.01.2012 | GESEG | 353 |
| | 27.01.2013 | RAKHEEG | 354 |

“X” indicating any amino acid

The epitopes identified by the inventors provide the most comprehensive data on potentially allergenic proteins and peptides derived from soybean that are available so far. These data, i.e. the identified epitopes, provide the essential basis for assessing both, the immune status of an individual to soybean protein and the allergenic potential of food and other products suspected of containing soybean protein. The compilation of the invention provides a collection of peptides of different amino acid sequences with each peptide comprising a short element of about three to six amino acids (sequence element), which corresponds to one of the identified epitopes (table 1). The sequence element may also have a length of about three to ten amino acids. Since the sequence element corresponds to one of the identified epitopes, each peptide presents a potential epitope for a soybean-allergen antibody (IgE). The term “corresponding” refers to the exact reproduction of the epitope sequence as well as variants thereof differing from the identified epitope in one or two amino acids. The variation may be due to one or two missing amino acids and/or to the exchange of one or two amino acids in comparison to the identified epitope. It is well established in the art that the secondary structure and the physical-chemical properties of a peptide (e.g. its antibody binding properties due to size, charge and/or polarity) may be maintained despite of minor changes to the amino acid sequence. In particular, single amino acids with side chains of little polarity or other reactivity, may be omitted or replaced by other amino acids of similar reactivity (e.g. lysine-arginine or leucine-isoleucine). The possibility of minor amino acid changes within the amino acid sequence corresponding to the epitope was also confirmed by the data obtained from the phage display and the peptide arrays. The antibodies contained in the patients' sera recognized several variants of individual epitopes, e.g. overlapping epitopes such as EDEEQQ (SEQ ID NO.: 32), EEQQRES (SEQ ID NO.: 33), RESEES (SEQ ID NO.: 34), (see table 1, and SEQ ID NO.: 385) of beta conglycinin alpha chain.

Accordingly, comprehensive analysis of interactions of antibody sera derived from various patients allowed to determine variations within the epitope sequences. Table 2 depicts the epitopes within the endogenous soybean protein sequence (epitopes underlined) and indicates the variability of the epitope (minimum amino acids/all amino acids of the epitope).

TABLE 2
Epitopes within the natural soybean protein

| | Epitope with adjacent amino | | |
|---|---|---|---|
| Soybean protein (Genebank | acids according to protein | SEQ ID | |
| ID) | sequence | NO. | variation |
| Defensin (Z13956.1) | VFASQVVVQTEGRVC | 355 | 4/6 |
| | VFASQVVVQTEGRVC | 356 | 4/6 |
| | VFASQVVVQTEGRVC | 357 | 4/6 |
| | QVVVQTEGRVCESQS | 358 | 4/7 |
| | QTEGRVCESQSHGFH | 359 | 4/5 |
| | RVCESQSHGFHGLCNR | 360 | 4/7 |
| | SQSHGFHGLCNRDHN | 361 | 4/9 |
| | GFHGLCNRDHNCALVC | 362 | 4/9 |
| | LCNRDHNCALVCRNE | 363 | 4/6 |
| | RCKRSRRCFCTRICG | 364 | 4/6 |
| beta conglycinin alpha chain | PKHNKCLQSCNSERD | 365 | 4/7 |
| (X17698.1) | PKHNKCLQSCNSERD | 366 | 4/5 |
| | SCNSERDSYRNQACH | 367 | 4/6 |
| | SCNSERDSYRNQACHA | 368 | 4/7 |
| | ERDSYRNQACHARCN | 369 | 4/6 |
| | LKVEKEECEEGEIPR | 370 | 4/7 |
| | KEECEEGEIPRPRPR | 371 | 4/6 |
| | KEECEEGEIPRPRPR | 372 | 4/6 |
| | IPRPRPRPQHPEREPQ | 373 | 4/9 |
| | RPRPQHPEREPQQPGE | 374 | 4/7 |
| | QHPEREPQQPGEKEE | 375 | 4/6 |
| | QHPEREPQQPGEKEE | 376 | 4/7 |
| | QPGEKEEDEDEQPRP | 377 | 4/9 |
| | EDEQPRPIPFPRPQP | 378 | 4/6 |
| | PRPIPFPRPQPRQEEE | 379 | 4/6 |
| | PQPRQEEEHEQREEQ | 380 | 5/8 |
| | WPRKEEKRGEKGSEE | 381 | 5/7 |
| | RPPHQKEERNEEEDE | 382 | (4/7 |
| | QKEERNEEEDEDEEQQ | 383 | 4/6 |
| | RNEEEDEDEEQQRES | 384 | 4/7 |
| | EDEDEEQQRESEESE | 385 | 4/7 |
| | KFEEINKVLFSREEG | 386 | 5/8 |
| | INKVLFSREEGQQQG | 387 | 4/7 |
| | SEDKPFNLRSRDPIY | 388 | 4/7 |
| | LSIVDMNEGALLLPHF | 389 | 4/5 |
| | ELVGLKEQQQEQQQE | 390 | 4/7 |
| | ELVGLKEQQQEQQQE | 391 | 4/7 |
| | LKEQQQEQQQEEQPLE | 392 | 4/6 |
| | LLKNQRESYFVDAQP | 393 | 5/8 |
| | LLKNQRESYFVDAQP | 394 | 4/6 |
| | PFLFGSNRFETLFKN | 395 | 4/5 |
| | RFETLFKNQYGRIRVL | 396 | 4/6 |
| | YRILEFNSKPNTLLLP | 397 | 4/6 |
| | TTYYVVNPDNNENLRL | 398 | 4/6 |
| | LRLITLAIPVNKPGR | 399 | 4/5 |
| | IPVNKPGRFESFFLSS | 400 | 4/6 |
| | SRNILEASYDTKFEE | 401 | 4/5 |
| | LFSREEGQQQGEQRLQE | 402 | 4/7 |
| | GEQRLQESVIVEISK | 403 | 4/5 |
| | LSKRAKSSSRKTISSE | 404 | 4/6 |
| | FFEITPEKNPQLRDLD | 405 | 4/6 |
| | SEQDIFVIPAGYPVVV | 406 | 4/6 |
| | QVQELAFPGSAQAVE | 407 | 4/6 |
| beta conglycinin alpha′ chain | RCNLLKVEEEEECEE | 408 | 4/6 |
| (M13759.1) | QIPRPRPQHPERERQ | 409 | 4/6 |
| | PRPQHPERERQQHGEK | 410 | 4/6 |
| | ERQQHGEKEEDEGEQP | 411 | 4/7 |
| | HGEKEEDEGEQPRPF | 412 | 4/8 |
| | GEQPRPFPFPRPRQP | 413 | 4/7 |
| | RPFPFPRPRQPHQEE | 414 | 4/6 |
| | QEEEHEQKEEHEWHR | 415 | 4/6 |
| | QEEEHEQKEEHEWHR | 416 | 4/5 |
| | GGKGSEEEQDEREHP | 417 | 5/9 |
| | HKQEKHQGKESEEEEE | 418 | 4/6 |
| | HKQEKHQGKESEEEEE | 419 | 4/5 |
| | HQGKESEEEEEDQDE | 420 | 4/7 |
| | ESEEEEEDQDEDEEQ | 421 | 5/9 |
| | KESQESEGSESQREP | 422 | 4/9 |
| | KESQESEGSESQREP | 423 | 4/6 |
| | NKRSQQLQNLRDYRI | 424 | 4/8 |
| | KPNTLLLPHHADADY | 425 | 4/6 |
| | FEEINKVLFGREEGQ | 426 | 4/6 |
| | AIVVLVINEGEANIE | 427 | 4/6 |
| | NIELVGIKEQQQRQQ | 428 | 4/6 |
| | QDIFVIPAGYPVMVN | 429 | 4/7 |
| | ESYFVDAQPQQKEEG | 430 | 4/6 |
| | ESYFVDAQPQQKEEG | 431 | 4/6 |
| | ESYFVDAQPQQKEEG | 432 | 4/5 |
| | PLLLLGVVFLASVSV | 433 | 4/5 |
| | HFNSKRFQTLFKNQY | 434 | 4/5 |
| | ITQRNPQLRDLDVFL | 435 | 4/5 |
| | HPRPHQPHQKEEEKH | 436 | 4/5 |
| | LVNNDDRDSYNLQSG | 437 | 4/5 |
| | QSGDALRVPAGTTFY | 438 | 4/6 |
| beta conglycinin beta chain | QRFNKRSPQLENLRD | 439 | 4/6 |
| (S44893) | RDSYNLHPGDAQRIP | 440 | 4/5 |
| | RDSYNLHPGDAQRIP | 441 | 4/6 |
| | SHNILETSFHSEFEE | 442 | 4/8 |
| | SHNILETSFHSEFEE | 443 | 4/6 |
| | LETSFHSEFEEINRV | 444 | 4/6 |
| | NRVLFGEEEEQRQQE | 445 | 4/5 |
| | NRVLFGEEEEQRQQE | 446 | 4/6 |
| | FGEEEEQRQQEGVIV | 447 | 4/6 |
| | AIVILVINEGDANIE | 448 | 4/5 |
| | LLGTVFLASVCVSLK | 449 | 4/6 |
| | FLLFVLSGRAILTLVN | 450 | 4/5 |
| | VNPHDHQNLKIIKLA | 451 | 4/6 |
| | AIPVNKPGRYDDFFLS | 452 | 4/6 |
| | EGVIVELSKEQIRQLS | 453 | 4/6 |
| | GALLLPHFNSKAIVIL | 454 | 4/6 |
| | QKQKQEEEPLEVQRYR | 455 | 4/6 |
| | NVVRQIERQVQELAFP | 456 | 4/5 |
| Glycinin G1 (AB113349.1) | FSGCCFAFSSREQPQ | 457 | 4/6 |
| | FSGCCFAFSSREQPQ | 458 | 4/5 |
| | CFAFSSREQPQQNEC | 459 | 4/9 |
| | RPSYTNGPQEIYIQQ | 460 | 4/6 |
| | RPSYTNGPQEIYIQQ | 461 | 4/7 |
| | GMIYPGCPSTFEEPQ | 462 | 4/7 |
| | GMIYPGCPSTFEEPQ | 463 | 4/8 |
| | STFEEPQQPQQRGQS | 464 | 4/5 |
| | STFEEPQQPQQRGQS | 465 | 4/6 |
| | EPQQPQQRGQSSRPQ | 466 | 4/6 |
| | PQQRGQSSRPQDRHQ | 467 | 4/6 |
| | RPQDRHQKIYNFREG | 468 | 4/5 |
| | REGDLIAVPTGVAWW | 469 | 4/5 |
| | REGDLIAVPTGVAWW | 470 | 4/5 |
| | NSLENQLDQMPRRFY | 471 | 4/5 |
| | AGNQEQEFLKYQQEQ | 472 | 4/6 |
| | AGNQEQEFLKYQQEQ | 473 | 4/5 |
| | GHQSQKGKHQQEEEN | 474 | 4/8 |
| | GHQSQKGKHQQEEEN | 475 | 4/6 |
| | KQIAKNLQGENEGED | 476 | 4/6 |
| | GLSVIKPPTDEQQQR | 477 | 4/6 |
| | IKPPTDEQQQRPQEE | 478 | 4/6 |
| | DEKPQCKGKDKHCQR | 479 | 4/6 |
| | DEKPQCKGKDKHCQR | 480 | 4/5 |
| | FGSLRKNAMFVPHYN | 481 | 4/8 |
| | NNPFKFLVPPQESQK | 482 | 4/6 |
| | NNPFKFLVPPQESQK | 483 | 4/6 |
| | NNKPFQCAGVALSRCT | 484 | 4/5 |
| | KHCQRPRGSQSKSRRN | 485 | 4/8 |
| | KNAMFVPHYNLNANSIIY | 486 | 4/6 |
| | LNGRALIQVVNCNGER | 487 | 4/5 |
| | QNFVVAARSQSDNFE | 488 | 4/5 |
| | ARSQSDNFEYVSFKT | 489 | 4/7 |
| Glycinin G2 (D00216.1) | FALREQAQQNECQIQ | 490 | 4/5 |
| | QIQKLNALKPDNRIE | 491 | 4/6 |
| | LNALKPDNRIESEGG | 492 | 4/6 |
| | FPGCPSTYQEPQESQ | 493 | 4/5 |
| | FPGCPSTYQEPQESQ | 494 | 4/6 |
| | ESQQRGRSQRPQDRH | 495 | 4/5 |
| | ESQQRGRSQRPQDRH | 496 | 4/6 |
| | QRPQDRHQKVHRFRE | 497 | 4/6 |
| | QRPQDRHQKVHRFRE | 498 | 4/6 |
| | QSQKGKQQEEENEGS | 499 | 4/6 |
| | AFGVNMQIVRNLQGE | 500 | 4/6 |
| | VTAPAMRKPQQEEDD | 501 | 4/6 |
| | PQQEEDDDDEEEQPQ | 502 | 4/6 |
| | QNIGQNSSPDIYNPQ | 503 | 4/5 |
| | NCNGERVFDGELQEG | 504 | 4/5 |
| | NCNGERVFDGELQEG | 505 | 4/6 |
| | MIFPGCPSTYQEPQES | 506 | 4/5 |
| | YQQQQQGGSQSQKGKQ | 507 | 4/5 |
| | NEEEDSGAIVTVKGG | 508 | 4/5 |
| | IVTVKGGLRVTAPAMR | 509 | 4/5 |
| | TICTMRLRQNIGQNS | 510 | 4/6 |
| | PALWLLKLSAQYGSLR | 511 | 4/6 |
| | PQNFAVAAKSQSDNFE | 512 | 4/6 |
| | KTNDRPSIGNLAGANS | 513 | 4/7 |
| | KNNNPFSFLVPPQESQR | 514 | 4/6 |
| Glycinin G3 (X15123.1) | SFREQPQQNECQIQR | 515 | 4/6 |
| | IQQGSGIFGMIFPGC | 516 | 4/6 |
| | STFEEPQQKGQSSRP | 517 | 4/6 |
| | SRPQDRHQKIYHFRE | 518 | 4/6 |
| | FREGDLIAVPTGFAY | 519 | 4/5 |
| | TNSFQNQLDQMPRRF | 520 | 4/5 |
| | LSVISPPTEEQQQRP | 521 | 4/6 |
| | EEQQQRPEEEEKPDC | 522 | 4/6 |
| | PDCDEKDKHCQSQSR | 523 | 4/6 |
| | PDCDEKDKHCQSQSR | 524 | 4/6 |
| | PDCDEKDKHCQSQSR | 525 | 4/6 |
| | HCQSQSRNGIDETIC | 526 | 4/6 |
| | HNIGQTSSPDIFNPQ | 527 | 4/8 |
| | CFAFSFREQPQQNECQIQ | 528 | 4/6 |
| | EFLQYQPQKQQGGTQS | 529 | 4/6 |
| | AFVVDRQIVRKLQGENE | 530 | 4/5 |
| | QTFNLRRQQARQVKNN | 531 | 4/6 |
| Glycinin G4 (X05652) | QLNNLNALEPDHRVE | 532 | 4/6 |
| | RNGLHSPSYSPYPRM | 533 | 4/6 |
| | RNGLHSPSYSPYPRM | 534 | 4/6 |
| | LGVAIPGCPETFEEP | 535 | 4/6 |
| | LGVAIPGCPETFEEP | 536 | 4/5 |
| | QQLQDSHQKIRHFNE | 537 | 4/6 |
| | QQLQDSHQKIRHFNE | 538 | 4/7 |
| | PDIEYPETMQQQQQQ | 539 | 4/6 |
| | QQQKSHGGRKQGQHQ | 540 | 4/6 |
| | NEDIAEKLESPDDER | 541 | 4/6 |
| | NEDIAEKLESPDDER | 542 | 4/6 |
| | LSVISPKWQEQQDED | 543 | 4/5 |
| | LSVISPKWQEQQDED | 544 | 4/5 |
| | SHPPRRPSHGKREQD | 545 | 4/6 |
| | SHPPRRPSHGKREQD | 546 | 4/6 |
| | RRPSHGKREQDEDED | 547 | 4/6 |
| | GKRNKTGQDEDEDED | 548 | 4/6 |
| | WRSKKTQPRRPRQEE | 549 | 4/6 |
| | KTQPRRPRQEEPRER | 550 | 4/6 |
| | KTQPRRPRQEEPRER | 551 | 4/6 |
| | HNAVTSYLKDVFRAI | 552 | 4/5 |
| | MIIIAQGKGALGVAI | 553 | 4/6 |
| | EEGGSVLSGFSKHFLA | 554 | 4/6 |
| | SSSKLNECQLNNLNA | 555 | 4/5 |
| Glycinin G5 (M10962.1) | CQLNNLNALEPDHRV | 556 | 4/6 |
| | NLNALEPDHRVESEG | 557 | 4/5 |
| | NRNGSHLPSYLPYPQ | 558 | 4/8 |
| | NRNGSHLPSYLPYPQ | 559 | 4/6 |
| | AIGFAFPGCPETFEK | 560 | 4/6 |
| | AIGFAFPGCPETFEK | 561 | 4/6 |
| | FNEGDVLVIPLGVPY | 562 | 4/6 |
| | TYNTGDEPVVAISPL | 563 | 4/5 |
| | RVFYLAGNPDIEHPE | 564 | 4/6 |
| | RVFYLAGNPDIEHPE | 565 | 4/6 |
| | RVFYLAGNPDIEHPET | 566 | 4/5 |
| | QQQKSHGGRKQGQHR | 567 | 4/5 |
| | SHGGRKQGQHRQQEE | 568 | 4/6 |
| | RKQGQHRQQEEEGGS | 569 | 4/5 |
| | EDTAEKLRSPDDERK | 570 | 4/5 |
| | EGGLSVISPKWQEQE | 571 | 4/6 |
| | EGGLSVISPKWQEQE | 572 | 4/6 |
| | SVISPKWQEQEDEDE | 573 | 4/6 |
| | EQEDEDEDEDEEYGR | 574 | 4/6 |
| | SHGKHEDDEDEDEEE | 575 | 4/6 |
| | HEDDEDEDEEEDQPR | 576 | 4/6 |
| | EDEDEEEDQPRPDHP | 577 | 4/6 |
| | EEEDQPRPDHPPQRP | 578 | 4/6 |
| | QPRPDHPPQRPSRPE | 579 | 4/6 |
| | DHPPQRPSRPEQQEP | 580 | 4/6 |
| | RPEQQEPRGRGCQTR | 581 | 4/5 |
| | CQGNAVFDGELRRGQ | 582 | 4/6 |
| | CQGNAVFDGELRRGQ | 583 | 4/6 |
| | THHNAVSSYIKDVFR | 584 | 4/6 |
| | VLSNSYNLGQSQVRQ | 585 | 4/6 |
| | VLSNSYNLGQSQVRQ | 586 | 4/5 |
| | KYQGNSGPLVNPGSG | 587 | 4/5 |
| | KYQGNSGPLVNPGSG | 588 | 4/6 |
| | LIETWNSQHPELQCAG | 589 | 4/6 |
| | TWNSQHPELQCAGVTV | 590 | 4/6 |
| | QHPELQCAGVTVSKRT | 591 | 4/5 |
| | QCAGVTVSKRTLNRN | 592 | 4/6 |
| | IVVQGKGAIGFAFPGC | 593 | 4/5 |
| | QSSRRGSRSQQQLQD | 594 | 4/5 |
| | ERKQIVTVEGGLSVI | 595 | 4/7 |
| | DEEYGRTPSYPPRRPSH | 596 | 4/7 |
| | QTRNGVEENICTMKLHE | 597 | 4/6 |
| | ENIARPSRADFYNPKA | 598 | 4/5 |
| | RVRVVNCQGNAVFDG | 599 | 4/6 |
| | FDGELRRGQLLVVPQN | 600 | 4/5 |
| | VVPQNPAVAEQGGEQ | 601 | 4/6 |
| | DVFRVIPSEVLSNSYN | 602 | 4/5 |
| | SEVLSNSYNLGQSQV | 603 | 4/6 |
| Profilin1 (AJ223981.1) | AYVDDHLLCGIEGNH | 604 | 4/8 |
| | GNHLTHAAIIGQDGS | 605 | 4/6 |
| | GNHLTHAAIIGQDGS | 606 | 4/6 |
| | THAAIIGQDGSVWLQ | 607 | 4/5 |
| | LIIGIYDEPMTPGQC | 608 | 4/5 |
| | DFPQFKPEEITAIMN | 609 | 4/6 |
| | LQSTDFPQFKPEEITA | 610 | 4/5 |
| | MNDFNEPGSLAPTGL | 611 | 4/7 |
| | KYMVIQGEPGAVIRGKK | 612 | 4/6 |
| | CNMVVERLGDYLIDQG | 613 | 4/5 |
| Profilin2 (AJ223982.) | MVVERPGDYLIDQGY | 614 | 4/5 |
| | MVVERPGDYLIDQGY | 615 | 4/6 |
| | DDHLLCDIEGNHLTHA | 616 | 4/6 |
| | IIGQDGSVWAQSTDFP | 617 | 4/5 |
| PR10 (X60043.1) | HKIESIDEANLGYSY | 618 | 4/6 |
| | HKIESIDEANLGYSY | 619 | 4/7 |
| | VENVEGNGGPGTIKK | 620 | 4/6 |
| | GVFTFEDEINSPVAPA | 621 | 4/6 |
| | FEDEINSPVAPATLYK | 622 | 4/6 |
| | GGAALPDTAEKITFDS | 623 | 4/5 |
| | LPDTAEKITFDSKLV | 624 | 4/7 |
| | AGPNGGSAGKLTVKYET | 625 | 4/5 |
| | LTVKYETKGDAEPNQ | 626 | 4/9 |
| 2s Albumin (AAB71140.1) | LLISLLFCIAHTCSASKWQ | 627 | 4/7 |
| | IAHTCSASKWQHQQDSC | 628 | 4/6 |
| | SKWQHQQDSCRKQLQG | 629 | 4/6 |
| | QHQQDSCRKQLQGVNL | 630 | 4/7 |
| | QDSCRKQLQGVNLTPCE | 631 | 4/7 |
| | QLQGVNLTPCEKHIMEK | 632 | 4/5 |
| | IMEKIQGRGDDDDDD | 633 | 4/7 |
| | NEGKDEDEEEEGHMQKC | 634 | 4/5 |
| | EEGHMQKCCTEMSEL | 635 | 4/6 |
| | MQKCCTEMSELRSPKC | 636 | 4/6 |
| | SPKCQCKALQKIMENQ | 637 | 4/9 |
| | QKIMENQSEELEEKQKKK | 638 | 4/6 |
| | INLATMCRFGPMIQCDL | 639 | 4/6 |
| | RFGPMIQCDLSSDD | 640 | 4/8 |
| 2s Albumin (BT091363.1) | IAHTCCASKWQQHQQES | 641 | 4/6 |
| | CCASKWQQHQQESCREQ | 642 | 4/7 |
| | QQHQQESCREQLKGINL | 643 | 4/6 |
| | KGINLNPCEHIMEKIQ | 644 | 4/6 |
| | CEHIMEKIQAGRRGED | 645 | 4/7 |
| | GRRGEDGSDEDHILIRT | 646 | 4/5 |
| | YIRKKEGKEEEEEGH | 647 | 4/6 |
| | RKKEGKEEEEEGHMQK | 648 | 4/5 |
| | MQKCCSEMSELKSPI | 649 | 4/7 |
| | MSELKSPICQCKALQKI | 650 | 4/10 |
| | QKIMDNQSEQLEGKEKKQ | 651 | 4/6 |
| | ELMNLAIRCRLGPMIG | 652 | 4/5 |
| | RLGPMIGCDLSSDD | 653 | 4/5 |
| 18 kD Seed Maturation Protein | ETATNIGASAKAGME | 654 | 4/10 |
| (AJ574791.1) | MEKTKATVQEKAERMTAR | 655 | 4/6 |
| | AERMTARDPVQKELAT | 656 | 4/6 |
| | DPVQKELATQKKEAKM | 657 | 4/5 |
| | KQAARQHNTAAKQSA | 658 | 4/6 |
| | TAGHMGHGHHTTGTGT | 659 | 4/6 |
| | TYSTTGEYGQPMGAHQ | 660 | 4/5 |
| | QPMGAHQTSAMPGHG | 661 | 4/9 |
| | SAMPGHGTGQPTGHVTE | 662 | 4/6 |
| | GQPTGHVTEGVVGSHP | 663 | 4/7 |
| | VTEGVVGSHPIGTNRGP | 664 | 4/8 |
| Albumin 1_(AJ574791.1) | EAADCNGACSPFEMPPCR | 665 | 4/7 |
| | SPFEMPPCRSRDCRCVP | 666 | 4/5 |
| | PPCRSRDCRCVPIGL | 667 | 4/7 |
| | RSRDCRCVPIGLVAGFC | 668 | 4/6 |
| | PIGLVAGFCIHPTGLS | 669 | 4/6 |
| | CIHPTGLSSVAKMIDE | 670 | 4/8 |
| | KMIDEHPNLCQSDDECMK | 671 | 4/6 |
| | HPNLCQSDDECMKKGS | 672 | 4/8 |
| | MKKGSGNFCARYPNNYID | 673 | 4/6 |
| Albumin 1 (AJ223037.1) | SPFEVPPCRSSDCRCV | 674 | 4/5 |
| | SVAKMVDEHPNLCQS | 675 | 4/5 |
| | GWCFDSDSEALKGFL | 676 | 4/9 |
| Albumin 1 | MQEGTSSAKLTTHLNK | 677 | 4/6 |
| (AJ223037.1_G3FGW7) | RSSDCRCVPIALFVGF | 678 | 4/6 |
| Seed biotinylated protein | EIHVEKHRVPKMATH | 679 | 4/6 |
| (U59626.1) | EIHVEKHRVPKMATH | 680 | 4/6 |
| | KDHAGKAMGDIGGRG | 681 | 4/6 |
| | HAAANVVGNKESQRE | 682 | 4/5 |
| | HAAANVVGNKESQRE | 683 | 4/6 |
| | ESGGQVVAEKGRETE | 684 | 4/5 |
| | ESGGQVVAEKGRETE | 685 | 4/6 |
| | AAAHVVEGAAGYAGH | 686 | 4/5 |
| | EYTAKKKEEAQRELE | 687 | 4/7 |
| | EYTAKKKEEAQRELE | 688 | 4/6 |
| | QPQEAEERPSEGIGE | 689 | 4/5 |
| | NTMGGESEGGGGKEE | 690 | |
| | VLETRVTGRAKHEEG | 691 | 4/6 |
| Lectin (K00821.1) | GLAFFLAPIDTKPQT | 692 | 4/5 |
| | GLAFFLAPIDTKPQT | 693 | 4/6 |
| | FLAPIDTKPQTHAGY | 694 | 4/7 |
| | FDTFRNSWDPPNPHI | 695 | 4/5 |
| | FDTFRNSWDPPNPHI | 696 | 4/6 |
| | ASNLPHASSNIDPLD | 697 | 4/6 |
| Gly m BD 28K (AB046874.2) | GGYVPCRQEEDEELHHKC | 698 | 4/6 |
| p34 thiol protease (J05560.1) | GVITQVKYQGGCGRG | 699 | 4/5 |
| Kunitz trypsin inhibitor | AFGGIRAAPTGNERC | 700 | 4/6 |
| (S45092.1) | AFGGIRAAPTGNERC | 701 | 4/6 |
| | AVIMLCVGIPTEWSV | 702 | 4/5 |
| | AVIMLCVGIPTEWSV | 703 | 4/6 |
| Hydrophobic Seed Protein | ILGGSLGTVDDCCAL | 704 | 4/5 |
| (A29385) | ILGGSLGTVDDCCAL | 705 | 4/6 |

In the compilation of the invention, at least five different epitopes are represented. The compilation of the invention is for example suitable for determining the immune status of a patient to soybean proteins by analysing the patient's immunological reaction to selected epitopes. As each peptide of the compilation presents a potentially allergenic soybean epitope, interaction of the patient's antibodies (IgE) with the peptides is representative for the patient's immune status to soybean allergens.

Additionally, a peptide comprising at least one sequence element corresponding to a soybean epitope is suitable for identifying or generating molecules specifically binding to the respective epitope and peptides or proteins comprising the same. The resulting molecules, e.g. antibodies, provide a significant advantage for detecting soybean proteins. Common anti-soybean antibodies or sera are raised against whole soybean proteins or even proteomes and therefore inevitably include antibodies directed against non-allergen proteins/parts of proteins. In contrast, utilizing peptides with sequence elements corresponding to specific allergenic soybean epitopes allows for the generation of antibodies distinctly directed against allergenic soybean proteins. Moreover, these antibodies specifically bind to the allergenic part (i.e epitope) of a soybean protein. Finally, such antibodies, or other binding molecules generated by using the peptides of the invention, allow the detection of allergenic remnants of soybean proteins that could be missed using antibodies binding to non-allergenic parts of a protein.

The term “compilation” refers to a collection of at least five different peptides, which includes that each peptide may be present in several or even hundreds of identical copies. Each peptide comprises at least one sequence element corresponding to an epitope, but may also comprise more, e.g. two or three sequence elements corresponding to identical and/or different epitopes. For example, each peptide may comprise multiple sequence elements corresponding to different epitopes. A peptide comprising sequence elements corresponding to the same epitope may comprise the same sequence element (thus representing the same epitope) but different additional amino acids. In this case, the same sequence element is presented in various different molecular environments, e.g. secondary structures. Therefore, it is to be understood that the compilation may comprise both, peptides comprising sequence elements corresponding to different epitopes as well as peptides comprising identical sequence elements, thus corresponding to the same epitope, but comprising different additional amino acids.

The compilation may be provided by immobilizing the peptides on a carrier, e.g. a chip, slide, well plate or on beads. Likewise, the compilation may be provided as a multitude of compositions, each composition containing identical copies of a peptide or a mixture of different peptides. Alternatively, the compilation may be provided as a single composition containing different peptides comprising sequence elements corresponding to the same or different epitopes, wherein at least five different epitopes are represented.

In a preferred embodiment, at least 10, preferably at least 20, more preferred at least 50 different epitopes are represented in the compilation.

In a preferred embodiment, each of the at least five different peptides comprises a sequence element corresponding to a different epitope.

In a preferred embodiment, the compilation comprises at least 10, preferably at least 20, more preferred at least 50 different peptides. Depending on the application, the advantageous number of different peptides may vary. For example, in a simple diagnostic application, a few peptides, e.g. five to ten peptides, may be sufficient to investigate sera for the general presence of antibodies against soybean proteins. In such cases, the sequence elements are preferably selected to correspond to epitopes of the most allergenic proteins such as Gly m Bd 28K, glycinin, hydrophobic seed protein (Gly m 1a), kunitz trypsin inhibitor, profilin (Gly m3), β-conglycinin (Gly m Bd 60K) and defensin (Gly 2m). For more detailed analyses, the compilation may comprise about 20 to 500 different peptides, preferably representing at least one epitope of each identified allergenic soybean protein. For example, such analysis could be used for providing a more personalized analysis of a patient's health condition. For such comprehensive analysis, a peptide array or a bead-based multiplex assay may be provided covering sequence elements corresponding to each of the identified epitopes.

In a preferred embodiment, the peptide consists of about 5 to 30 amino acids, preferably 8 to 20 amino acids, more preferred 8 to 15 amino acids. Besides the sequence element corresponding to the epitope, usually four, five or six amino acids, each peptide may comprise further amino acids on one or both sides of the sequence element. Such additional amino acids may be added for example for immobilizing the peptide on a substrate or for embedding the sequence element corresponding to the epitope into a specific molecular environment (secondary structure). The additional amino acids may be chosen depending on the use of the compilation. For example, to present the epitope in its natural molecular environment, the sequence element corresponding to the epitope may be flanked by amino acids as found in the naturally occurring protein sequence of the soybean protein. In case the epitope is flanked by different amino acid sequences in different soybean varieties, corresponding peptides may be used to evaluate the influence of the adjacent amino acids to the allergenic potential of the epitope. Additionally, functional molecules, e.g. for coupling or readout, may be added at the N- or C-terminus of the peptide.

In a preferred embodiment, each epitope is selected from one of the groups comprising group 1 (β-conglycinin) consisting of SEQ ID NO.: 1-93, group 2 (defensin) consisting of SEQ ID NO.: 94-103, group 3 (Gly m Bd 28K) consisting of SEQ ID NO.: 104, group 4 (glycinin) consisting of SEQ ID NO.: 105-250, group 5 (hydrophobic seed protein) consisting of SEQ ID NO.: 251-253, group 6 (kunitz trypsin inhibitor) consisting of SEQ ID NO.: 254-257, group 7 (P34 thiol protease) consisting of SEQ ID NO.:258-259, group 8 (profilin) consisting of SEQ ID NO.: 260-274, and group 9 (Pr-10) consisting of SEQ ID NO.: 275-283. Although the sensitivity to soybean proteins is known to vary distinctly between different patients, several proteins are considered major allergens, in particular P34 (Gly ml), β-conglycinin (Gly m5) and glycinin (Gly m6). β-conglycinin and glycinin are storage proteins and constitute 70 to 80% of total seed protein. Both are regarded good diagnostic markers for soybean protein sensitivity (Masilamani et al., 2012). β-conglycinin comprises three individual subunits (α, α′, β), which were all found to react with IgE. Interestingly, wild and cultivated soybean plants were found to vary distinctly in these subunits (Natarajan 2014). Glycinin comprises five subunits, which were also all found to be recognized by IgE from soybean protein sensitive persons. P34 is a soybean vacuole protein (hull protein) with high sequence similarity to thiol proteases of the papain family, which are known to potentially induce allergic reactions in human. In general, several soybean allergens show distinct similarity to allergens of other organisms. For example, P34 is related to DERp1 from dust mite, profilin (Gly m3) is related to the profilin family of proteins in birch pollen, Gly m5/6 is closely related to ARAh1/3 of peanut and Pr10 (Gly m4) is highly homolog to BETV1 and other major allergens of cross reactivity with birch pollen. Thus, compilations covering sequence elements corresponding to epitopes of the above mentioned groups are suitable to provide detailed information not only on soybean sensitivity but also regarding sensitivity to allergens from other organisms. In particular, when providing screening tests to identify soybean protein sensitive patients, the compilation of peptides may cover at least five, preferably all of these proteins. Accordingly, in a preferred embodiment, each of the at least five different epitopes is derived from a different soybean protein. Thus, each epitope is preferably selected from a different group of the groups comprising group 1 (β-conglycinin) consisting of SEQ ID NO.: 1-93, group 2 (defensin) consisting of SEQ ID NO.: 94-103, group 3 (Gly m Bd 28K) consisting of SEQ ID NO.: 104, group 4 (glycinin) consisting of SEQ ID NO.: 105-250, group 5 (hydrophobic seed protein) consisting of SEQ ID NO.: 251-253, group 6 (kunitz trypsin inhibitor) consisting of SEQ ID NO.: 254-257, group 7 (P34 thiol protease) consisting of SEQ ID NO.:258-259, group 8 (profilin) consisting of SEQ ID NO.: 260-274, and group 9 (Pr-10) consisting of SEQ ID NO.: 275-283.

In a preferred embodiment, at least nine different epitopes are represented in the compilation, wherein each epitope is selected from a different group of the groups comprising group 1 (β-conglycinin) consisting of SEQ ID NO.: 1-93, group 2 (defensin) consisting of SEQ ID NO.: 94-103, group 3 (Gly m Bd 28K) consisting of SEQ ID NO.: 104, group 4 (glycinin) consisting of SEQ ID NO.: 105-250, group 5 (hydrophobic seed protein) consisting of SEQ ID NO.: 251-253, group 6 (kunitz trypsin inhibitor) consisting of SEQ ID NO.: 254-257, group 7 (P34 thiol protease) consisting of SEQ ID NO.:258-259, group 8 (profilin) consisting of SEQ ID NO.: 260-274, and group 9 (Pr-10) consisting of SEQ ID NO.: 275-283.

In a particularly preferred embodiment, the compilation comprises at least nine different peptides and each peptide comprising at least one sequence element corresponding to an epitope selected from each of the groups comprising group 1 (β-conglycinin) consisting of SEQ ID NO.: 1-93, group 2 (defensin) consisting of SEQ ID NO.: 94-103, group 3 (Gly m Bd 28K) consisting of SEQ ID NO.: 104, group 4 (glycinin) consisting of SEQ ID NO.: 105-250, group 5 (hydrophobic seed protein) consisting of SEQ ID NO.: 251-253, group 6 (kunitz trypsin inhibitor) consisting of SEQ ID NO.: 254-257, group 7 (P34 thiol protease) consisting of SEQ ID NO.:258-259, group 8 (profilin) consisting of SEQ ID NO.: 260-274, and group 9 (Pr-10) consisting of SEQ ID NO.: 275-283.

In a preferred embodiment, each epitope is selected from group 10 consisting of SEQ ID NO.: 18, 22, 29, 34, 37, 60, 65, 67, 79, 85, 87, 91, 99, 95, 94, 92, 103, 117, 131, 110, 126, 152, 153, 168, 173, 172, 183, 188, 200, 227, 233, 237, 250, 251, 252, 253, 254, 255, 257, 258, 262, 271, 261, 276, 281, 274, 309, 311, 319, 307, 294, 305, 298, 284, 291, 290, 328, 322, 323, 331, 332, 333, 334, 335, 336, 337, 339, 340, 345, 346 and 351. Although the immune status to major allergenic soybean proteins varies distinctly between patients, statistic analyses enabled by the use of the specific phage display library, made it possible to identify certain epitopes that are recognized by the majority of patients (Table 3).

TABLE 3
Epitopes recognized by the majority of patients:

| | | SEQ ID |
|---|---|---|
| Protein | Epitope | NO. |
| beta conglycinin alpha | QACHAR | 18 |
| chain (X17698) | RPQHPEREP | 22 |
| | EEHEQ | 29 |
| | EEQQRES | 34 |
| | EEGQQQG | 37 |
| beta conglycinin | SEEEQDERE | 60 |
| alpha′ chain (M13759.1) | ESQESEGSE | 65 |
| | NKRSQQLQ | 67 |
| beta conglycinin beta chain | IERQVQ | 79 |
| (S44893) | EEINRV | 85 |
| | ELSKEQ | 87 |
| | EEEPLE | 91 |
| Defensin (Z13956.1) | HGLCNRDHN | 99 |
| | RVCESQS | 95 |
| | TEGRVC | 94 |
| | VFASQV | 92 |
| Gly m BD 28K | QEEDEE | 103 |
| Glycinin G1 (AB113349.1) | PQQPQQR | 117 |
| | QQRPQE | 131 |
| | SREQPQ | 110 |
| | KYQQEQ | 126 |
| Glycinin G2 (D00216.1) | GRSQR | 152 |
| | QDRHQK | 153 |
| | QQKGQS | 168 |
| Glycinin G3 (X15123.1) | QQQRPE | 173 |
| | PTEEQ | 172 |
| Glycinin G4 (X05652) | RNGLHS | 183 |
| | IRHFNE | 188 |
| | PRQEEP | 200 |
| Glycinin G5 (M10962.1) | IEHPET | 227 |
| | KWQEQE | 233 |
| | EEEDQP | 237 |
| Hydrophobic Seed Protein | LGGSL | 250 |
| (A29385) | DDCCAL | 251 |
| | NSCGRS | 252 |
| Kunitz trypsin inhibitor | GNERC | 253 |
| (S45092.1) | RAAPTG | 254 |
| | LCVGIP | 255 |
| p34 thiol protease (J05560.1) | VITQVK | 257 |
| | KYQGGC | 258 |
| Profilin (AJ223981.1) | QGEPGAV | 262 |
| | GSVWAQ | 271 |
| | EPGSL | 261 |
| PR10 (X60043.1) | VENVEGN | 276 |
| | GSAGKLT | 281 |
| | SIDEA | 274 |
| 2s Albumin (BT091363.1) | QHQQES | 309 |
| | ESCREQL | 311 |
| | NQSEQLEGKE | 319 |
| 2s Albumin (BT091363.1) | IQCDLS | 307 |
| | LFCIAHTCS | 294 |
| | NQSEELEEK | 305 |
| | KQLQGVN | 298 |
| 18 kD Seed Maturation Protein | ATVQEKAER | 284 |
| (AJ574791.1) | HGTGQPTGH | 291 |
| | HQTSA | 290 |
| Albumin 1_(AJ574791.1) | HPNLCQSD | 328 |
| | NGACSPFE | 322 |
| | PPCRSRD | 323 |
| Albumin 1 (AJ223037.1) | PPCRSS | 331 |
| | VDEHP | 332 |
| | SDSEA | 333 |
| Albumin 1 (AJ223037.1) | EGTSSAKLT | 334 |
| | RCVPIA | 335 |
| Lectin (K00821.1) | LAPIDT | 336 |
| | TKPQT | 337 |
| | DPPNPHI | 339 |
| | DTFRN | 340 |
| Seed biotinylated protein | KESQRE | 345 |
| (U59626.1) | ANVVG | 346 |
| | EAQRELE | 351 |

These epitopes are particularly preferred for providing a general immune status test, because it can be expected that every sensitive person will respond to at least one of these epitopes. For example the epitopes RNQACHA (SEQ ID NO.: 17), EIPRP (SEQ ID NO.: 20), NSERD (SEQ ID NO.: 15) have been recognized by 15/23 patients resp. 3/23 resp 7/23. All three epitopes have been recognized just by one patient, two epitopes (SEQ ID NO.: 15, SEQ ID NO.: 17) by 5/23 patients.

In a further aspect, the invention relates to an analysis tool comprising at least five different peptides, each peptide comprising at least one sequence element corresponding to an epitope selected from the group consisting of SEQ ID NO.: 1-354, wherein at least five different epitopes are represented. The term “analysis tool” as used herein refers to means and products for chemical, biochemical or medical analysis of samples, e.g. biological samples. Such tools include devices (e.g. microarray, multi-well plates), as well as reagents (e.g. compositions comprising free or bead-bound peptides) suitable for single or multiplex analyses. When presented on the surface of a chip or beads, the peptides can interact with substances, e.g. other molecules, brought into contact with the surface. In a peptide array, for example, multitudes of identical peptides are deposited in spots, each spot representing a different peptide. In case of a bead based analysis tool, identical peptides may be conjugated to the surface of a single bead. The peptides can be attached to a surface using hydrophilic linker moieties to avoid sterical hindrance, which might influence the interaction between the peptide and the sample. The analysis tools of the invention are for example suitable for diagnostic applications such as determining a patient's immune status to soybean proteins. Serum derived from a person suspected of being sensitive to soybean protein is brought into contact with the analysis tool to allow the IgE or other antibody classes of the patient to interact with and bind to the peptides. The binding of the antibodies can be specifically detected and analyzed. Depending on the aim of the diagnosis, the analysis tool may cover epitopes of different soybean allergens. For example, for a general initial screening, the analysis tool may contain peptides comprising sequence elements corresponding to epitopes of some or all major allergens of soybean. Additionally, the analysis tool may contain peptides comprising sequence elements corresponding to epitopes of allergens of other organisms to provide a more comprehensive survey. For a more detailed analysis, all major soybean allergens should be represented by the analysis tool. When used in analysis tools such as microarrays or bead based assays, the peptides preferably consist of 5 to 30, more preferred of 8 to 20, most preferred of 8 to 15 amino acids.

In a further aspect, the invention relates to an in vitro method for determining a patient's immune status to soybean allergens, comprising the steps providing at least five different peptides, each peptide comprising at least one sequence element corresponding to an epitope selected from the group consisting of SEQ ID NO.: 1-354, wherein at least five different epitopes are represented, contacting each peptide with a sample comprising antibodies derived from the patient, and detecting an interaction of each peptide with the sample. The term “immune status” refers to the presence of antibodies in the blood of a person, in particular to the presence of antibodies directed to soybean proteins. The method allows revealing whether a patient produces antibodies recognizing soybean proteins and, thus, is expected to show allergic reactions when consuming soybean containing products and at least one of these antibodies is an IgE antibody. Moreover, the method is suitable to specifically identify the epitopes of soybean proteins, to which the patient reacts. This may provide further information about the strength of the allergy and may indicate whether the patient is likely to react to allergens from other organisms as well, since related allergenic epitopes are found in proteins of various organisms. Dependent on the distinct aim of using the method, various numbers of peptides comprising sequence elements corresponding to at least five different epitopes may be employed. For example, to merely test whether a person in question is likely to show any allergic reaction to soybean proteins, a few peptides (e.g. five to ten) representing epitopes of at least two, preferably five major allergenic proteins (epitopes of groups 1 to 9) may be sufficient. For a more detailed analysis on the antibody status of the patient, peptides comprising sequence elements corresponding to epitopes selected from all major soybean allergens, preferably from all potentially allergenic soybean proteins, may be covered. The results of such a comprehensive analysis can help to decide about a patient's personalized treatment, e.g. form the basis for a subsequent desensitization protocol.

For carrying out the method, the peptides can be provided as a microarray or bead-based multiplex assay. For a simpler approach, the method may be used in form of a lateral flow test. In any case, the peptides are incubated with the patient's sample such that the molecules contained in the sample, in particular the patient's antibodies, can interact and bind to the peptides. Binding of the antibodies is subsequently detected, e.g. using secondary antibodies directed against human immunoglobulins. In case the secondary antibodies are fluorescently labelled, the analysis can be performed using fluorescence scanners and suitable data analysis software, which allows for a fast and standardized readout providing reliable results in reasonable time.

In a preferred embodiment, the sample is a body fluid sample, preferably a blood sample, more preferred a serum sample. For analyzing a patient's immune status to soybean allergens, antibodies derived from the patient are contacted with peptides comprising sequence elements corresponding to the identified epitopes. The antibodies may be derived from the patient through a blood or serum sample, which are usually used for testing on IgE. Serum samples are particularly preferred since they are devoid of several blood compounds that could interfere with peptide binding. Moreover, blood shows high autofluorescence and is therefore disadvantageous in case a fluorescent readout is used. In addition, samples derived from lymphocyte transformation tests may be used (Pichler and Tilch, 2004).

Alternatively to determining the patient's immune status using a body fluid sample, allergenic sensitivity is commonly tested using skin allergy tests such as skin prick test, skin scratch test, intrademic test or patch test. In all these tests the potential allergen is applied onto or beneath the patient's skin. In case the patient is sensitive to the allergen, i.e. produces antibodies recognizing the allergen, the patient will develop rash and/or urticaria in the proximity of the applied allergen. Since the peptides of the invention comprise sequence elements corresponding to epitopes that can be specifically recognized by IgE of soybean protein sensitive patients, they are suitable for respective allergy tests. Accordingly, in a further aspect, the invention relates to a method for determining a patient's sensitivity to soybean allergens, comprising the steps providing a compound comprising at least five different sequence elements each corresponding to an epitope selected from the group consisting of SEQ ID NO.: 1-354, wherein at least five different epitopes are represented, contacting the patient with the compound, preferably by applying the compound to and/or beneath the patient's skin, and detecting a reaction, preferably rash and/or urticaria. Upon application, e.g. beneath the patient's skin, the epitope is recognized by IgE antibodies localized on mast cells, inducing dimerisation of the IgE receptors and subsequent degranulation of the mast cell. Thus, for application in allergy tests, at least two peptides comprising at least five different sequence elements corresponding to epitopes, preferably derived from the same allergenic protein, are combined into a single compound. This can be achieved, for example, by providing a polypeptide comprising the sequence elements corresponding to epitopes, e.g. a homo- or heterodimer or polymer, respectively. Alternatively, the compound may be provided by associating the peptides with a cargo molecule. Suitable cargo molecules include chemical compounds as well as surface molecules such as nanoparticles (e.g. iron oxide nanoparticles or albumin nanoparticles), liposomes, microparticles or microbubbles.

In a further aspect, the invention relates to a kit comprising at least one composition, containing a compound comprising at least five different sequence elements each corresponding to an epitope selected from the group consisting of SEQ ID NO.: 1-354, wherein at least five different epitopes are represented. The composition of the kit may represent various epitopes of a single soybean protein by containing compounds that comprise various sequence elements corresponding to different epitopes of the soybean protein of interest. The various sequence elements may be comprised in a single compound or allocated to different compounds. The composition of the kit may also cover epitopes of several or even all allergenic soybean proteins. The kit is suitable to provide potential soybean protein allergens for various applications, in particular skin allergy tests. Accordingly, the composition may be a composition suitable for diagnostic tests such as skin prick test or patch test. The concentration, in which the peptides are provided and applied to the skin, should be according to biological standardization, e.g histamine equivalent potency (HEP/ml) or allergy units (AU/ml) as defined by the Food and Drug Administration of the United States.

In a further aspect, the invention relates to a method for detecting at least one soybean allergen in a substance, comprising the steps providing at least two different peptides, each peptide comprising at least one sequence element corresponding to an epitope selected from the group consisting of SEQ ID NO.: 1-354, raising at least one antibody against each peptide, contacting the substance with the antibodies, and determining binding of the antibodies to the substance. Detection of the soybean allergen may include a qualitative detection and/or a quantitative analysis of the amount of soybean protein present in the substance. In a first step, epitopes of interest are selected, preferably representing one or more of the major allergenic soybean proteins. Depending on the aim of the analysis, different epitopes of various soybean products may be covered. To gain comprehensive information on the presence of potentially allergenic proteins in a sample, e.g. of a food product, at least one epitope of each major allergenic protein, preferably of each potentially allergenic protein should be detected. To exclude the presence of any soybean remnants e.g. for baby nutrition, the method may be performed using antibodies for the most frequently recognized or each of the identified epitopes. Accordingly, depending on the number of epitopes and allergens to be covered by the method, at least 5 different peptides, preferably at least 10 different peptides, more preferred at least 20 different peptides, most preferred at least 50 different peptides are provided and antibodies raised against them. In a preferred embodiment, at least five different epitopes, preferably at least 10 different epitopes, more preferred at least 20 different epitopes, most preferred at least 50 different epitopes are represented by the peptides. In a preferred embodiment, each peptide comprises a sequence element corresponding to a different epitope. Finally, epitopes may be used that are recognized by most patients (group 10, table 3) giving a good first impression on the immune status of the patient.

After selecting the epitopes, peptides are produced comprising at least one of the selected epitopes. Each peptide may consist of the epitope sequence(s) or may comprise additional amino acids on one or both sides of the epitope(s). The additional amino acids may for example correspond to the amino acids flanking the epitope in the natural protein or in the original peptide identified by the phage display. Alternatively, they may be individually selected, e.g. to optimize antibody formation. For example, to avoid hindrance of interactions between the peptide and an antibody, flanking amino acids may be selected having small residues, such as glycine, alanine or serine. The size of the peptide amounts preferably to 5 to 30, more preferred to 8 to 20, most preferred to 8 to 15 amino acids. For raising antibodies, preferably monoclonal antibodies, methods well known in the art may be employed. To detect soybean proteins or protein remnants in a substance, the substance is contacted with the antibodies to allow interaction and binding, which is then determined or even quantified, e.g. by use of fluorescent secondary antibodies and fluorescence scanners. In summary, the method allows determining the allergenic potential of the substance by detecting allergenic proteins or remnants of soybean proteins therein.

In a preferred embodiment, the substance is a human or animal food product, a dietary supplement or a cosmetic product. Soybean protein and preparations thereof are not only processed in human and animal food products but also in dietary supplements and cosmetics, e.g. in the form of soybean extracts or soybean oil. Since allergy reactions do not only occur upon oral ingestion but also upon contact to skin, a test for potentially allergenic proteins and protein remnants is likewise needed for cosmetic products and ingredients.

In a preferred embodiment, the soybean allergen is selected from the group consisting of profilin, P34 thiol protease, kunitz trypsin inhibitor, hydrophobic seed protein, glycinin, Gly m BD 28K, defensin, β-glycinin and PR10. These major allergenic soybean proteins are known to induce strong allergic reactions and are thus considered most crucial. Therefore, preferably each of these proteins is represented by at least one epitope. For testing products with particularly strict safety requirements, e.g. baby nutrition, all epitopes of the major allergenic soybean proteins or even of all potentially allergenic soybean proteins (Table 1) may be detected.

In a further aspect, the invention relates to a method for producing an immunoassay product, comprising the steps providing at least two different peptides, each peptide comprising at least one sequence element corresponding to an epitope selected from the group consisting of SEQ ID NO.: 1-354, raising at least one antibody against each peptide, and compiling the antibodies to provide the immunoassay product. Processed food and other industrial products contain soybean protein in various quantities and different conditions. For example, processing of soybean seeds or extracted protein leads to denaturation and modification of the soybean proteins, which can eliminate epitopes and thereby reduce the allergenic potential of the product. Therefore, methods are already used and continuously developed that aim on reducing the allergenic soybean proteins, e.g. by food processing techniques (Wilson et al., 2005) or genetic modification of soybean plants (WO 2010/087888). Moreover, wild (glycine soja) and cultivated (glycine max) soybean plants as well as different varieties of glycine max were found to have different variants of potentially allergenic proteins. The proteins differ at least slightly in their amino acid sequences and therefore vary in their allergenic potential. This prompted the development of specific glycine max varieties, which express reduced amounts of potentially allergenic proteins, e.g. P34 (Joseph et al., 2006). Despite these efforts, a reliable and complete destruction or removal of all allergenic proteins from soybean products has not yet been achieved. Therefore, it is necessary to assess the remaining allergenic potential of food and other products comprising soybean proteins. So far, soybean protein tests are mainly based on antibodies raised against the complete soybean proteome. Sometimes polyclonal antibodies are even directly derived from animals or humans sensitive to soybean proteins. However, such antibodies or sera do not necessarily provide reliable results. Antibodies usually only recognize the particular protein or amino acid residues they have been raised against. Therefore, antibodies raised against native proteins may no longer recognize soybean proteins having an altered confirmation due to industrial processing techniques. Thus, the allergenic epitope, although present, may not be bound by the respective antibodies. Moreover, antibodies or sera raised against the entire proteome of a specific variety may fail to detect allergenic proteins of a different variety. In contrast, antibodies raised against individual and defined epitopes of about four to ten amino acids have a high potential to recognize allergenic proteins or amino acid residues independently of conformational changes in the molecule (e.g. due to food processing techniques) or adjacent amino acid sequences (that may vary according to soybean variety). Thus, the identified epitopes and peptides comprising sequence elements corresponding to these epitopes are particularly suitable for providing an immunoassay product for detecting potentially allergenic soybean proteins. The term “antibody” as used herein refers to natural as well as artificial immunoglobulins and derivatives thereof, including polyclonal antibodies, monoclonal antibodies, antibody fragments, antigen-binding fragments (Fab) and single chain variable fragments (scFv).

For providing an immunoassay product, the peptides and antibodies may be selected and provided as described above. Depending on the number of epitopes and allergens to be detected by the immunoassay product, at least 5 different peptides, preferably at least 10 different peptides, more preferred at least 20 different peptides, most preferred at least 50 different peptides are provided and antibodies raised against them. In a preferred embodiment, at least five different epitopes, preferably at least 10 different epitopes, more preferred at least 20 different epitopes, most preferred at least 50 different epitopes are represented by the peptides. In a preferred embodiment, each peptide comprises a sequence element corresponding to a different epitope. The antibodies are compiled to provide the immunoassay product, which is suitable for testing a sample for the presence of soybean proteins, e.g. by contacting the sample with the antibodies and detecting possible interactions.

In a preferred embodiment, compiling the antibodies comprises immobilizing them onto a solid surface, e.g a chip, a multiwell plate or beads. Thereby, they can be easily contacted with the sample to be tested and the non-bound remnants of the sample removed by washing if necessary. This allows a fast and specific detection suitable to be implied into industrial processes.

In a preferred embodiment, the immunoassay product is a microarray, a bead-based assay product, an ELISA plate or a lateral flow test. Microarrays and bead-based assay products are particularly preferred for detecting the presence of a multitude of different epitopes, because they allow the simultaneous use and specific readout of many antibodies directed against different epitopes. ELISA (enzyme-linked immunosorbent assay) plates, usually multi-well or microtiter plates, also allow for a simultaneous testing of a substantial number of different antibodies. Lateral flow tests, in contrast, provide a particular fast readout, however, usually cover only one or few different antibodies. Depending on the number of epitopes to be detected and the time available, different immunoassay products may be advantageous. All of the assays are fully established standard methods allowing a sensitive and fast readout of antibody-antigen interaction. Moreover, readout systems have been developed, in particular for microarrays and ELISA that allow a fast and fully automated analysis.

In a further aspect, the invention relates to a method for determining the allergenicity of a soybean variety by detecting the presence of at least two epitopes in a sample of the variety, wherein the epitopes are selected from the group consisting of SEQ ID NO.: 1-354. In addition to wild (glycine soja) and cultivated (glycine max) soybean, several varieties of glycine max are known. Additionally, plant varieties expressing reduced levels of allergenic proteins (e.g. P34, Joseph et al., 2006) or alternative amino acid sequences of the allergenic proteins (e.g. WO 2010/087888) were specifically developed to reduce the allergenic potential. By detecting the presence and, preferably, also the quantity of the allergenic epitopes or proteins, the allergenicity of a particular soybean variety can be determined. This is of particular importance, since the use of soybean varieties with reduced allergenicity could contribute to a total reduction of newly developed allergies.

In a preferred embodiment, the presence of at least five different epitopes, preferably at least 10 different epitopes, more preferred at least 20 different epitopes, most preferred at least 50 different epitopes is detected.

In a preferred embodiment, the presence of the epitopes is detected by an immunoassay or mass spectrometry. Suitable immunoassays comprise, for example, ELISA and flow rate assays that may be provided by raising antibodies specific to the identified epitopes. In addition, mass spectrometry can be used to directly analyze the proteome of soybean varieties for the presence or the quantity of epitopes and the corresponding soybean proteins, respectively (e.g. Houston et al., 2011).

In a further aspect, the invention relates to the use of a peptide comprising a sequence element corresponding to an epitope selected from the group consisting of SEQ ID NO.: 1-354 for providing a molecule binding to a protein or peptide comprising the epitope, in particular an antibody. The term “molecule binding to a protein or peptide comprising the epitope” refers to any molecule that is able to specifically form a complex with the epitope and/or a peptide or protein containing a sequence element corresponding to the epitope due to intermolecular forces. Peptides consisting of, or comprising one or more sequence elements corresponding to the epitopes of a specific soybean protein may be used to identify molecules interacting and binding to the epitope and the original protein, e.g. by screening molecule libraries. Likewise, the peptides may be used to raise antibodies specifically recognizing and binding the epitope. Thus, in contrast to commonly used anti-soybean antibodies or sera, the molecules provided by using the peptides of the invention, specifically detect the epitope(s) of the protein responsible for allergic reactions to soybean. Accordingly, the binding molecules are particularly suitable for detecting allergenic proteins of soybean, for example in food or cosmetic products. In order to test the binding specificity of a molecule binding to a protein or peptide comprising the epitope, in particular an antibody, a compilation comprising at least five different peptides, each comprising the same sequence element corresponding to the epitope but different additional amino acids, may be used.

In a further aspect, the invention relates to a method for generating a variant of an allergenic soybean protein having a reduced allergenic potential, comprising the steps providing an amino acid sequence of at least one epitope selected from the group consisting of SEQ ID NO.: 1-354, altering the amino acid sequence of the at least one epitope as to eliminating the structure of the epitope, and generating a protein or peptide comprising the altered amino acid sequence. The term “variant of an allergenic soybean protein” refers to a variant of a soybean protein, which is known to induce allergenic reactions, that comprises one or more epitopes that have a different amino acid sequence compared to the natural protein. The term “a protein having reduced allergenic potential” also comprises proteins having no allergenic potential at all. The identified soybean epitopes (Tables 1 and 2) are suitable to generate variants of natural soybean proteins with an amino acid sequence that is altered in a way that binding of IgE antibodies of a soybean sensitive patient do not bind or bind to a reduced extent.

This can be achieved by replacing or removing amino acids within one or more of the identified epitopes (table 1), which are essential for IgE binding. For example, to support the selection of suitable residues, epitopes from the group consisting of SEQ ID NO.: 1-354 can be mapped onto existing structures of the respective soy protein, or structures of homologous variants of the protein from other, favourably closely related, species available in the protein structure databases. One or more selected amino acids within the epitope's sequence may be replaced by amino acids of different physiochemical properties, such as size, charge or polarity. As a result, the modified epitope, and more importantly, proteins comprising the modified epitope are not recognized by epitope specific antibodies. In case an allergenic soybean protein was found to comprise several epitopes, one or more epitopes may be eliminated. For example, to determine the patient's antibody specificity, all epitopes found to induce cross-reactivity with antibodies against proteins of other species (e.g. birch pollen) may be eliminated.

Proteins with one or more epitopes eliminated, are suitable for analysing antibody-epitope interaction, as they allow determining the participation of individual amino acids in this interaction. Additionally, they provide important negative controls for analysis tools based on epitope specific antibodies. By including respective variants of soybean proteins, antibody binding and specificity can be evaluated in detail.

In addition, the method can be employed for generating derivatives of natural soybean proteins comprising one or more modified epitopes. Such derivates are, for example, suitable for immunotherapeutic applications. Modified allergens, i.e. derivatives of allergens suitable to provoke an IgG immunoresponse, but lacking the allergenic epitopes responsible for IgE reactivity, can be used as recombinant vaccines to reduce or even prevent allergenic reactions (Valenta and Niederberger, 2007).

In a further aspect, the invention relates to a peptide or protein comprising a modified variant of at least one epitope selected from the group consisting of SEQ ID NO.: 1-354, wherein the original structure of the epitope was eliminated.

### EXAMPLES

Affinity Selection and Panning

24 μg of serum protein from patients with soy allergy were diluted in 2.5 ml PBS and coated in protein binding Immuno™ Tubes with Maxisorp™ for 1 h at 4° C. at 18 rpm (negative control: PBS). The coated tubes were washed with 2.5 ml blocking buffer (5% NFDM/1×PBS) and incubated with 4 ml blocking buffer at 4° C. for at least 15 min (18 rpm). The tubes were washed three times with 4 ml wash buffer I (1×PBS). 1 ml blocking buffer containing 30 μg of non-allergic serum protein and the phage library ENTE1 was added to each tube. The tubes were incubated for 2 h at 4° C. (18 rpm). After incubation, the tubes were washed 5 times with 1 ml 0.1% Tween/1×PBS and subsequently with 0.5% Tween/1×PBS. Then the bound phages were eluted using 1 ml of elution buffer (0.1 M Glycin.HCl pH 2.2). The tubes were incubated 5 min and vortexed several times. The eluate was immediately neutralized with 200 μl neutralization buffer (1 M Tris.HCl pH 8.2). In the first selection round 4×10¹¹ cfu of the ENTE1-library were used per tube. In the second selection round 1000×the recovered phages were inserted.

Infection of TG1 (λ) with Eluted Phage

TG1 (λ) cells were grown in 250 ml baffled culture flasks in 10 ml dYT at 37° C. until they reached an OD₆₀₀ about 0.5-1.0. The cells were incubated with eluted phage for 20 min at 37° C. with 100 rpm agitation. After incubation, the solution was immediately poured on dYT-agar plates (25×25 cm) containing 200 μg/ml ampicillin and incubated overnight at 30° C.

Packaging of Phage from Infected Cells

After overnight incubation the colonies were resuspended in 25 ml dYT containing 1×10¹⁰ cfu/ml M13K07. The cells were diluted in 25 ml dYT containing 500 μg/ml ampicillin and 1×10¹⁰ cfu/ml M13K07 using a 250 ml baffled culture flasks to obtain a cell density of OD₆₀₀ 0.5. The cells were incubated for 1 h at 37° C. with 180 rpm agitation. Afterwards the culture was growing about 13 h at 30° C. with 180 rpm agitation.

Purification of Phage

The infected bacteria cells were centrifuged at 20,000×g for 20 min at 4° C. ¼ volume of cold PEG/NaCl (PEG/NaCl (20% (w/v) PEG 6000, 2.5 M NaCl) solution was added to the supernatant and incubated for at least 1 h on ice. Phages were collected by centrifugation (30 min, 4° C., 14,000×g) and the pellet was resuspended in 1 ml 1×PBS. The solution was centrifuged at 14,000×g for 10 min and the supernatant recovered and incubated with IA volume of PEG/NaCl (PEG/NaCl (20% (w/v) PEG 6000, 2.5 M NaCl) solution for at least 20 min on ice. Phages were again collected by 30 min centrifugation at 14,000×g, 4° C. and the pellet of phage particles resuspended in 1 ml PBS. The purified phage suspension was stored at 4° C. for several days.

Identification of Selected Epitopes

For data analysis the LibDB software was used. Due to the design of the trinucleotide based library, sequences with potential errors were removed. All valid sequences were written into a database and a database of 3-5mer motifs was generated. The observed frequency as well as statistically and theoretically expected values were provided for each motif in the database.

Validation of Epitopes Using a Peptide Array

The peptide micro arrays were designed to represent nine predominant soybean allergens, named beta-conglycinin, glycinin, defensin, lectin, PR-10, Gly m 1, P34, Kunitz trypsin-inhibitor and profilin. Each allergen amino acid sequence was divided into 15mer fragments with 4 amino acids overlap and a chip was designed bearing duplicates of every peptide. The slides were purchased from the company PEPperPRINT, which synthesized the peptides in a cycle of synthesis, where amino acid micro particles are printed directly on a glass slide based on solid phase Fmoc chemistry.

To analyze the IgE reactivity of soybean allergic patients to the soybean antigen peptides displayed on the array, the glass slides were prepared as follows. Each slide was washed in staining buffer (PBS, 0.05% (v/v) Tween 20, 0.1% (w/v) BSA, pH 7.4) for 10 min at room temperature and afterwards blocked for 30 min in blocking buffer (PBS, 0.05% (v/v) Tween 20, 1% (w/v) BSA, pH 7.4). Incubation of the slides in patient sera diluted 1:50 in staining buffer was performed over night at 4° C. After 3 times washing in standard buffer (PBS, 0.05% (v/v) Tween 20, pH 7.4) the slides were shaken at 200 rpm for 30 min in a goat-anti human IgE antibody solution (Invitrogen; 1:5000 diluted in staining buffer). Detection was performed by incubating the array after 3 additional washing steps in a solution containing the secondary donkey-anti goat antibody labelled with Cy5 (Abcam, 1:5000 diluted in staining buffer) for another 30 min at room temperature. The fluorescence signals on the array were detected in a micro array reader at 635 nm.

Data evaluation was performed using the PepSlide Analyser software from PEPperPRINT, where signal intensity was directly connected to the peptide spot. Additionally the software in a first step performed the subtraction of the local background signal for every spot, resulting in a normalization of the different background levels for the tested sera. Further for all sera an overall threshold level from 30 fluorescence signal intensity units was set, where signal intensities above this threshold were assumed as positively recognized by the patient sera. Peptides detected by a minimum of 25% of the 16 analyzed patient sera were defined as soybean related allergenic epitopes. Peptide sequences tested positive for IgE binding (as a measure of allergy relevance) in a peptide array were entered in the software and matched/aligned with the motives in the database. A sequence was considered to represent a potential epitope, if the enrichment value of the 4mer motives was different in comparison with the naïve library. The enrichment value of the motive in the serum was higher than the enrichment value of the motive in the naïve library. All sequences with the identified motive were saved in a fastq-file, which was aligned to soybean proteins using the software MegAlign Pro 12. If the 4mer motive could be expanded to a 5mer or 6mer motive, the potential epitope was validated for inclusion into table 1.

Identification of Novel Epitopes

Sequences of soybean proteins were matched with the motives in the database and the statistic listed. Then, a graph of enrichment values of the naïve library and the serum was generated in Excel. A positive difference of the values of the naïve library and the serum indicated a relevant epitope. In the second panning more than one sequence should be enriched. All sequences with the identified motive were saved in a fastq-file, which was aligned to soybean proteins using the software MegAlign Pro 12. If the 4mer motive could expand to a 5mer or 6mer motive, the potential epitope was validated.

For example, the motive “QHQQ” was identified in the first panning contained in

>2013_S1_2. Panning_seq_10 Icount=250: GIEPCSKYSQHQQHVQN (SEQ ID NO.: 706) and

>2013_S1_2. Panning_seq_11 Icount=122: GEIMCWREVVTQHQQHA (SEQ ID NO.: 707).

In the second panning, the motive was significantly enriched with a total of 825 counts in comparison to 407 counts in the first panning (Serum 1_1. Panning round: count 407, Serum 1_2. Panning round: count 825).

Validation of an Epitope Using Magnetic Beads

200 μl Dynabeads® M-270 Carboxylic Acid (Invitrogen) were washed 3 times with 1 ml PBS. The beads were activated by adding of 200 μl 0.4 M 1-ethyl-3-(3-dimethyl-aminopropyl) carbodiimide (EDC) and 200 μl 0.1 M N-hydroxysuccinimide (NHS). After 5 minute incubation at room temperature with agitation, the beads were washed once with 1 ml 10 mM sodium acetat, pH 4.5. 100 μg streptavidin in 100 μl 10 mM sodium acetat, pH 4.5 were added. After 30 min incubation at room temperature with agitation, beads were blocked with 500 μl 0.1 M ethanolamine pH 8.5 for 10 minutes at room temperature with agitation. The beads were washed 3 times with 1 ml PBS and resuspended in 200 μl PBS.

Potential epitopes were synthesized as peptides and coupled with 10 μl streptavidin coupled beads by adding of 3 μg (=3 μl) peptide for 1 h at room temperature with agitation. Beads were blocked with 500 μl blocking buffer (15 mM Tris, 0.14 M NaCl, 1% BSA, 0.05% Tween) for 30 minutes at room temperature with agitation. After three washing steps with 1 ml PBS, the beads were incubated with 10 μl serum from a patient with soy allergy for 1 h at room temperature with agitation and afterwards washed 3 times with 1 ml 0.1% Tween/PBS and resuspended in 10 μl SDS-loading Dye.

Immediately a SDS-Page with a 12% gel at 180 V for 50 minutes was performed. The western blot was performed with 0.2 μm nitrocellulose membrane and 260 mA and 200 V for 2 h at 4° C. After blotting the membrane was blocked with 0.1% Tween/PBS for 1 hour at room temperature. The antibodies (Anti-human polyvalent immunoglobulins) were diluted 1:10000 in 10 ml 0.1% Tween/PBS. After incubation for 1 h at room temperature, three washing steps with 0.1% Tween/PBS and one with PBS were performed. Antibodies were detected using DAB-solution (10 ml 0.1m Imidazole, pH 7, 50 μl 1% Diaminobenzidine, 5 μl 30% H₂O₂).

In Vitro Analysis of the Immune Status of Soybean-Sensitive Patients Using a Peptide Microarray

Epitope candidates were supplied externally (Neundorf, University of Cologne, Germany). The peptide is synthesised with the following structure: Ac-N-epitope sequence-C-Ebes-Lys(γ-N₃)-amide (Ebes=amino-3,6-dioxaoctyl-succinamic acid). The peptide sequences were based on either the naïve soybean protein sequence (Pr) or the mimotope from phage display experiment (Ph). The peptide length varied between eight and nineteen amino acids. Glass slides were coated with Dibenzocyclooctyl (DBCO). The peptides were immobilized using different concentrations (81 ng/μl, 27 ng/μl or 9 ng/μl) on glymo-covered slides using click chemistry.

Each slide was blocked for 30 min with PBS containing 0.1% (v/v) Tween-20 and 1% (w/v) BSA, pH 7.4. For the incubation with sera cover slides (HybriWell™. 60 mm×21 mm×0.15) were used. Sera from 50 patients with differently intense prick reaction to soy milk were tested. The slides were incubated with 100 μl sera from the soybean-sensitive patients diluted 1:50 in blocking buffer overnight at 4° C. After two washes in washing buffer (PBS containing 0.1% (v/v) Tween-20, pH 7.4) the slides were shaken at 200 rpm for 60 min in a mouse-anti human IgE antibody (Abcam, Cambridge, UK, ab99834) diluted to 100 ng/ml or goat-anti human IgG antibody Cy5 (Abcam, Cambridge, UK, ab97172) diluted to 100 ng/ml in blocking buffer. After two further washes, bound IgG and IgE were detected by incubating the array for 60 min at room temperature with the secondary Cy5-labeled goat anti-mouse antibody (life technologies, Thermo Fisher Scientific, Waltham, Mass., USA, A10524) diluted to 400 ng/ml in blocking buffer. Fluorescence signals on the array were detected in a microarray reader (Geneprix 4200A) at 635 nm and the data were evaluated using GenePix® Pro 6 software to determine the signal intensity of each peptide spot. The software calculated the signal to noise ratio (SNR) for every spot. An overall threshold of 5 signal to noise ratio units was set, and signals above this threshold were assumed to indicate positive recognition by the patient sera.

TABLE 4
Peptide sequences used in the microarray

| | | SEQ ID | | |
|---|---|---|---|---|
| | | NO. of | | Peptide sequence (sequence element |
| Protein | source | epitope | Peptide | corresponding to epitope underlined) |
| Gly m 2 | Ph | 100 | P1 | GFNLCNRDRPAP (SEQ ID NO.: 734) |
| Gly m 2 | Pr | 100 | P2 | SQSHGFHGLCNRDHNCA (SEQ ID |
| | | | | NO.: 735) |
| Gly m 2 | Ph | 94 | P3 | EVSFASQVLIY (SEQ ID NO.: 736) |
| Gly m 5.01 | Ph | 28 | P4 | SHHDQPRQHA (SEQ ID NO.: 737) |
| Gly m 5.01 | Ph | 35 | P5 | HITFSREEGSI (SEQ ID NO.: 738) |
| Gly m 5.01 | Pr | 35 | P6 | KVLFSREEGQQQ (SEQ ID NO.: 739) |
| Gly m 5.02 | Pr | 54 | P7 | DEGEQPRPFPFPRPR (SEQ ID NO.: |
| | | | | 740) |
| Gly m 5.02 | Ph | 50 | P8 | HIQEEECEGDL (SEQ ID NO.: 741) |
| Gly m 5.02 | Pr | 50 | P9 | VEEEEECEEGQI (SEQ ID NO.: 742) |
| Gly m 6.01 | Ph | 127 | P10 | SDKYQEEFQPR (SEQ ID NO.: 743) |
| Gly m 6.01 | Pr | 127 | P11 | TEKYQQNSSG (SEQ ID NO.: 744) |
| Gly m 6.01 | Pr | 127 | P12 | EFLKYQQEQG (SEQ ID NO.: 745) |
| Gly m 6.02 | Ph | 159 | P13 | GVYNSQVDDEEEQNQRD (SEQ ID |
| | | | | NO.: 746) |
| Gly m 6.03 | Ph | 126 | P14 | HVVEQEFLD (SEQ ID NO.: 747) |
| Gly m 6.03 | Pr | 126 | P15 | RRFYLAGNQEQEFL (SEQ ID NO.: |
| | | | | 748) |
| Gly m 6.03 | Ph | 154 | P16 | ISQQDRHRI (SEQ ID NO.: 749) |
| Gly m | Ph | 345 | P17 | FDQKESQHFS (SEQ ID NO.: 750) |
| Agglutinin | | | | |
| Gly m | Pr | 345 | P18 | HAANVVGNKESQREAR (SEQ ID NO.: |
| Agglutinin | | | | 751) |
| Gly m BD | Ph | 104 | P19 | GYNPCRQEEDEELHHKC (SEQ ID |
| 28K | | | | NO.: 752) |
| Gly m BD | Ph | 104 | P20 | QDQEEDEEDE (SEQ ID NO.: 753) |
| 28K | | | | |

TABLE 5
Recognition of peptides by patient sera

| | Number of IgE- | Number of IgG- | |
|---|---|---|---|
| Peptide | positive sera | positive sera | total |
| P1 | 4 | 4 | 8 |
| P2 | 7 | 1 | 8 |
| P3 | 6 | 3 | 9 |
| P4 | 12 | 6 | 18 |
| P5 | 12 | 2 | 14 |
| P6 | 21 | 9 | 31 |
| P7 | 2 | 2 | 4 |
| P8 | 2 | 2 | 4 |
| P9 | 32 | 17 | 50 |
| P10 | 10 | 1 | 12 |
| P11 | 2 | 3 | 5 |
| P12 | 12 | 12 | 24 |
| P13 | 7 | 1 | 8 |
| P14 | 23 | 5 | 28 |
| P15 | 3 | 2 | 6 |
| P16 | 28 | 13 | 42 |
| P17 | 0 | 1 | 2 |
| P18 | 8 | 1 | 9 |
| P19 | 9 | 6 | 15 |
| P20 | 5 | 2 | 7 |

The results show that the peptides are recognized by antibodies in the sera of soybean-protein sensitive patients. The results further show that the sensitivity to different soybean proteins and epitopes varies.

In contrast to commonly used methods for determining the immune status of a patient, the method disclosed facilitates a differentiation between IgE and IgG antibodies.

### REFERENCES

EP 14 166 662.8
WO 2010/087888
Joseph L M, Hymowitz T, Schmidt M A, and Herman E M. Evaluation of glycine germplasm for nulls of the immunodominant allergen P34/GlymBd30k. Crop Sci. 2006 46, 1755-1763.
Houston N L, Lee D G, Stevenson S E, Ladics G S, Bannon G A, McClain S, Privalle L, Stagg N, Herouet-Guicheney C, Macintosh S C, Thelen J J. Quantitation of soybean allergens using tandem mass spectrometry. J Proteome Res. 2011 Feb. 4; 10(2):763-73.
Masilamani M, Commins S, Shreffler W. Determinants of food allergy. Immunol Allergy Clin North Am. 2012 February; 32(1):11-33.
Natarajan S S. Analysis of Soybean Seed Proteins Using Proteomics. Journal of Data mining in Genomics and Proteomics. 2014, 5:1.
Pichler W J, Tilch J. Allergy. 2004 August; 59(8):809-20.
Valenta R, Niederberger V. Recombinant allergens for immunotherapy. J Allergy Clin
Immunol. 2007 April; 119(4):826-30. Epub 2007 Mar. 1.
Wilson S, Blaschek K, de Mejia E. Allergenic proteins in soybean: processing and reduction of P34 allergenicity. Nutr Rev. 2005 February; 63(2):47-58.
Yang W W, Gonzales de Mejia E, Zheng H, and Lee Y. Soybean Allergens: Presence, Detection and Methods for Mitigation in Soybean and Health, InTech 2011, ISBN 978-953-307-535-8.

1. A compilation comprising at least five different peptides, each peptide comprising at least one sequence element corresponding to an epitope selected from the group consisting of SEQ ID NO.: 50, 154, 35, 127, 104, 1-34, 36-49, 51-103, 105-126, 128-153 and 155-354, wherein at least five different epitopes are represented.

2. The compilation of claim 1, wherein each of the at least five different peptides comprises a sequence element corresponding to a different epitope.

3. The compilation of claim 1 or 2, wherein the compilation comprises at least 10, preferably at least 20, more preferred at least 50 different peptides.

4. The compilation of any one of claims 1 to 3, wherein each epitope is selected from one of the groups comprising
group 1 (β-conglycinin) consisting of SEQ ID NO.: 1-93,
group 2 (defensin) consisting of SEQ ID NO.: 94-103,
group 3 (Gly m Bd 28K) consisting of SEQ ID NO.: 104,
group 4 (glycinin) consisting of SEQ ID NO.: 105-250,
group 5 (hydrophobic seed protein) consisting of SEQ ID NO.: 251-253,
group 6 (kunitz trypsin inhibitor) consisting of SEQ ID NO.: 254-257,
group 7 (P34 thiol protease) consisting of SEQ ID NO.:258-259,
group 8 (profilin) consisting of SEQ ID NO.: 260-274, and
group 9 (Pr-10) consisting of SEQ ID NO.: 275-283.

5. The compilation of any one of claims 1 to 4, wherein each epitope is selected from group 10 consisting of SEQ ID NO.: 18, 22, 29, 34, 37, 60, 65, 67, 79, 85, 87, 91, 99, 95, 94, 92, 103, 117, 131, 110, 126, 152, 153, 168, 173, 172, 183, 188, 200, 227, 233, 237, 250, 251, 252, 253, 254, 255, 257, 258, 262, 271, 261, 276, 281, 274, 309, 311, 319, 307, 294, 305, 298, 284, 291, 290, 328, 322, 323, 331, 332, 333, 334, 335, 336, 337, 339, 340, 345, 346 and 351.

6. An in vitro method for determining a patient's immune status to soybean allergens, comprising the steps
providing at least five different peptides, each peptide comprising at least one sequence element corresponding to an epitope selected from the group consisting of SEQ ID NO.: 50, 154, 35, 127, 104, 1-34, 36-49, 51-103, 105-126, 128-153 and 155-354, wherein at least five different epitopes are represented,
contacting each peptide with a sample comprising antibodies derived from the patient, and
detecting an interaction of each peptide with the sample.

7. The method of claim 6, wherein the sample is a body fluid sample, preferably a blood sample, more preferred a serum sample.

8. A kit comprising at least one composition containing a compound comprising at least five different sequence elements each corresponding to an epitope selected from the group consisting of SEQ ID NO.: 50, 154, 35, 127, 104, 1-34, 36-49, 51-103, 105-126, 128-153 and 155-354, wherein at least five different epitopes are represented.

9. A method for detecting at least one soybean allergen in a substance, comprising the steps
providing at least two different peptides, each peptide comprising at least one sequence element corresponding to an epitope selected from the group consisting of SEQ ID NO.: 50, 154, 35, 127, 104, 1-34, 36-49, 51-103, 105-126, 128-153 and 155-354,
raising at least one antibody against each peptide,
contacting the substance with the antibodies, and
determining binding of the antibodies to the substance.

10. A method for producing an immunoassay product, comprising the steps
providing at least two different peptides, each peptide comprising at least one sequence element corresponding to an epitope selected from the group consisting of SEQ ID NO.: 50, 154, 35, 127, 104, 1-34, 36-49, 51-103, 105-126, 128-153 and 155-354,
raising at least one antibody against each peptide, and
compiling the antibodies to provide the immunoassay product.

11. The method of claim 10, wherein compiling the antibodies comprises immobilising the antibodies onto a solid surface.

12. The method of claim 10 or 11, wherein the immunoassay product is a microarray, a bead-based assay product, an ELISA plate or a lateral flow test.

13. A method for determining the allergenicity of a soybean variety by detecting the presence of at least two epitopes in a sample of the variety, wherein the epitopes are selected from the group consisting of SEQ ID NO.: 50, 154, 35, 127, 104, 1-34, 36-49, 51-103, 105-126, 128-153 and 155-354.

14. The method of claim 13, wherein the presence of the epitopes is detected by an immunoassay or mass spectrometry.

15. Use of a peptide comprising a sequence element corresponding to an epitope selected from the group consisting of SEQ ID NO.: 50, 154, 35, 127, 104, 1-34, 36-49, 51-103, 105-126, 128-153 and 155-354 for providing a molecule binding to a protein or peptide comprising the epitope.

16. A method for generating a variant of an allergenic soybean protein having a reduced allergenic potential, comprising the steps
providing an amino acid sequence of at least one epitope selected from the group consisting of SEQ ID NO.: 50, 154, 35, 127, 104, 1-34, 36-49, 51-103, 105-126, 128-153 and 155-354,
altering the amino acid sequence of the at least one epitope as to eliminating the structure of the epitope, and
generating a protein or peptide comprising the altered amino acid sequence.


SEQUENCE LISTING (753 sequences)

SEQ ID NO: 1 - protein, 5 aa, glycine max
SNRFE

SEQ ID NO: 2 - protein, 6 aa, glycine max
FKNQYG

SEQ ID NO: 3 - protein, 6 aa, glycine max
FNSKPN

SEQ ID NO: 4 - protein, 6 aa, glycine max
VNPDNN

SEQ ID NO: 5 - protein, 5 aa, glycine max
LAIPV

SEQ ID NO: 6 - protein, 8 aa, glycine max
  SITE 2..2: Xaa may be any amino acid
NXPGRFES

SEQ ID NO: 7 - protein, 5 aa, glycine max
EASYD

SEQ ID NO: 8 - protein, 6 aa, glycine max
EQQQGE

SEQ ID NO: 9 - protein, 5 aa, glycine max
QESVI

SEQ ID NO: 10 - protein, 6 aa, glycine max
KSSSRK

SEQ ID NO: 11 - protein, 6 aa, glycine max
PEKNPQ

SEQ ID NO: 12 - protein, 6 aa, glycine max
FVIPAG

SEQ ID NO: 13 - protein, 5 aa, glycine max
AFPGS

SEQ ID NO: 14 - protein, 7 aa, glycine max
CLQSCNS

SEQ ID NO: 15 - protein, 5 aa, glycine max
NSERD

SEQ ID NO: 16 - protein, 6 aa, glycine max
ERDSYR

SEQ ID NO: 17 - protein, 7 aa, glycine max
RNQACHA

SEQ ID NO: 18 - protein, 6 aa, glycine max
QACHAR

SEQ ID NO: 19 - protein, 7 aa, glycine max
KEECEEG

SEQ ID NO: 20 - protein, 6 aa, glycine max
EIPRPR

SEQ ID NO: 21 - protein, 6 aa, glycine max
KEECEE

SEQ ID NO: 22 - protein, 9 aa, glycine max
RPQHPEREP

SEQ ID NO: 23 - protein, 8 aa, glycine max
REPQQPGE

SEQ ID NO: 24 - protein, 6 aa, glycine max
HPEREP

SEQ ID NO: 25 - protein, 7 aa, glycine max
QQPGEKE

SEQ ID NO: 26 - protein, 9 aa, glycine max
EDEDEQPRP

SEQ ID NO: 27 - protein, 8 aa, glycine max
IPFPRPQP

SEQ ID NO: 28 - protein, 6 aa, glycine max
PRQEEE

SEQ ID NO: 29 - protein, 9 aa, glycine max
EEHEQREEQ

SEQ ID NO: 30 - protein, 7 aa, glycine max
GEKGSEE

SEQ ID NO: 31 - protein, 7 aa, glycine max
RNEEEDE

SEQ ID NO: 32 - protein, 6 aa, glycine max
EDEEQQ

SEQ ID NO: 33 - protein, 7 aa, glycine max
EEQQRES

SEQ ID NO: 34 - protein, 6 aa, glycine max
RESEES

SEQ ID NO: 35 - protein, 8 aa, glycine max
VLFSREEG

SEQ ID NO: 36 - protein, 7 aa, glycine max
EEGQQQG

SEQ ID NO: 37 - protein, 8 aa, glycine max
LRSRDPIY

SEQ ID NO: 38 - protein, 5 aa, glycine max
LSIVD

SEQ ID NO: 39 - protein, 7 aa, glycine max
LKEQQQE

SEQ ID NO: 40 - protein, 5 aa, glycine max
EQQQE

SEQ ID NO: 41 - protein, 6 aa, glycine max
EEQPLE

SEQ ID NO: 42 - protein, 8 aa, glycine max
NQRESYFV

SEQ ID NO: 43 - protein, 6 aa, glycine max
FVDAQP

SEQ ID NO: 44 - protein, 5 aa, glycine max
GVVFL

SEQ ID NO: 45 - protein, 5 aa, glycine max
RFQTL

SEQ ID NO: 46 - protein, 5 aa, glycine max
PQLRD

SEQ ID NO: 47 - protein, 5 aa, glycine max
QPHQK

SEQ ID NO: 48 - protein, 5 aa, glycine max
DRDSY

SEQ ID NO: 49 - protein, 5 aa, glycine max
LRVPA

SEQ ID NO: 50 - protein, 6 aa, glycine max
KVEEEE

SEQ ID NO: 51 - protein, 6 aa, glycine max
PERERQ

SEQ ID NO: 52 - protein, 6 aa, glycine max
QQHGEK

SEQ ID NO: 53 - protein, 7 aa, glycine max
EDEGEQP

SEQ ID NO: 54 - protein, 8 aa, glycine max
EGEQPRPF

SEQ ID NO: 55 - protein, 7 aa, glycine max
FPRPRQP

SEQ ID NO: 56 - protein, 6 aa, glycine max
QPHQEE

SEQ ID NO: 57 - protein, 6 aa, glycine max
EQKEEH

SEQ ID NO: 58 - protein, 5 aa, glycine max
KEEHE

SEQ ID NO: 59 - protein, 9 aa, glycine max
SEEEQDERE

SEQ ID NO: 60 - protein, 6 aa, glycine max
HKQEKH

SEQ ID NO: 61 - protein, 5 aa, glycine max
ESEEE

SEQ ID NO: 62 - protein, 7 aa, glycine max
EEEDQDE

SEQ ID NO: 63 - protein, 10 aa, glycine max
EEDQDEDEEQ

SEQ ID NO: 64 - protein, 9 aa, glycine max
ESQESEGSE

SEQ ID NO: 65 - protein, 6 aa, glycine max
ESQREP

SEQ ID NO: 66 - protein, 8 aa, glycine max
NKRSQQLQ

SEQ ID NO: 67 - protein, 6 aa, glycine max
LPHHAD

SEQ ID NO: 68 - protein, 6 aa, glycine max
GREEGQ

SEQ ID NO: 69 - protein, 6 aa, glycine max
VLVINE

SEQ ID NO: 70 - protein, 6 aa, glycine max
IELVGI

SEQ ID NO: 71 - protein, 7 aa, glycine max
QDIFVIP

SEQ ID NO: 72 - protein, 6 aa, glycine max
DAQPQQ

SEQ ID NO: 73 - protein, 7 aa, glycine max
LEVRKYR

SEQ ID NO: 74 - protein, 6 aa, glycine max
ESYFVD

SEQ ID NO: 75 - protein, 6 aa, glycine max
FVDAQP

SEQ ID NO: 76 - protein, 5 aa, glycine max
FLASV

SEQ ID NO: 77 - protein, 6 aa, glycine max
LSGRAI

SEQ ID NO: 78 - protein, 5 aa, glycine max
HQNLK

SEQ ID NO: 79 - protein, 6 aa, glycine max
KPGRYD

SEQ ID NO: 80 - protein, 6 aa, glycine max
ELSKEQ

SEQ ID NO: 81 - protein, 6 aa, glycine max
IERQVQ

SEQ ID NO: 82 - protein, 6 aa, glycine max
PQLENL

SEQ ID NO: 83 - protein, 6 aa, glycine max
DSYNLH

SEQ ID NO: 84 - protein, 5 aa, glycine max
PGDAQ

SEQ ID NO: 85 - protein, 6 aa, glycine max
SHNILE

SEQ ID NO: 86 - protein, 8 aa, glycine max
SFHSEFEE

SEQ ID NO: 87 - protein, 6 aa, glycine max
EEINRV

SEQ ID NO: 88 - protein, 6 aa, glycine max
RVLFGE

SEQ ID NO: 89 - protein, 5 aa, glycine max
QRQQE

SEQ ID NO: 90 - protein, 6 aa, glycine max
QEGVIV

SEQ ID NO: 91 - protein, 6 aa, glycine max
ILVINE

SEQ ID NO: 92 - protein, 6 aa, glycine max
PHFNSK

SEQ ID NO: 93 - protein, 6 aa, glycine max
EEEPLE

SEQ ID NO: 94 - protein, 6 aa, glycine max
VFASQV

SEQ ID NO: 95 - protein, 6 aa, glycine max
VVVQTE

SEQ ID NO: 96 - protein, 6 aa, glycine max
TEGRVC

SEQ ID NO: 97 - protein, 7 aa, glycine max
RVCESQS

SEQ ID NO: 98 - protein, 5 aa, glycine max
QSHGF

SEQ ID NO: 99 - protein, 7 aa, glycine max
SQSHGFH

SEQ ID NO: 100 - protein, 9 aa, glycine max
HGLCNRDHN

SEQ ID NO: 101 - protein, 9 aa, glycine max
RDHNCALVC

SEQ ID NO: 102 - protein, 6 aa, glycine max
LVCRNE

SEQ ID NO: 103 - protein, 5 aa, glycine max
SRRCF

SEQ ID NO: 104 - protein, 6 aa, glycine max
QEEDEE

SEQ ID NO: 105 - protein, 6 aa, glycine max
QCAGVA

SEQ ID NO: 106 - protein, 6 aa, glycine max
PRGSQS

SEQ ID NO: 107 - protein, 8 aa, glycine max
VPHYNLNA

SEQ ID NO: 108 - protein, 6 aa, glycine max
LIQVVN

SEQ ID NO: 109 - protein, 5 aa, glycine max
AARSQ

SEQ ID NO: 110 - protein, 5 aa, glycine max
DNFEY

SEQ ID NO: 111 - protein, 6 aa, glycine max
SREQPQ

SEQ ID NO: 112 - protein, 6 aa, glycine max
CCFAFS

SEQ ID NO: 113 - protein, 6 aa, glycine max
PQQNEC

SEQ ID NO: 114 - protein, 9 aa, glycine max
GPQEIYIQQ

SEQ ID NO: 115 - protein, 6 aa, glycine max
RPSYTN

SEQ ID NO: 116 - protein, 7 aa, glycine max
CPSTFEE

SEQ ID NO: 117 - protein, 7 aa, glycine max
IYPGCPS

SEQ ID NO: 118 - protein, 7 aa, glycine max
PQQPQQR

SEQ ID NO: 119 - protein, 5 aa, glycine max
QRGQS

SEQ ID NO: 120 - protein, 6 aa, glycine max
QSSRPQ

SEQ ID NO: 121 - protein, 6 aa, glycine max
PQDRHQ

SEQ ID NO: 122 - protein, 6 aa, glycine max
QKIYNF

SEQ ID NO: 123 - protein, 5 aa, glycine max
REGDL

SEQ ID NO: 124 - protein, 7 aa, glycine max
VPTGVAW

SEQ ID NO: 125 - protein, 5 aa, glycine max
NQLDQ

SEQ ID NO: 126 - protein, 5 aa, glycine max
QEQEF

SEQ ID NO: 127 - protein, 6 aa, glycine max
KYQQEQ

SEQ ID NO: 128 - protein, 5 aa, glycine max
HQSQK

SEQ ID NO: 129 - protein, 8 aa, glycine max
KHQQEEEN

SEQ ID NO: 130 - protein, 6 aa, glycine max
KNLQGE

SEQ ID NO: 131 - protein, 6 aa, glycine max
TDEQQQ

SEQ ID NO: 132 - protein, 6 aa, glycine max
QQRPQE

SEQ ID NO: 133 - protein, 6 aa, glycine max
EKPQCK

SEQ ID NO: 134 - protein, 6 aa, glycine max
DKHCQR

SEQ ID NO: 135 - protein, 5 aa, glycine max
FVPHY

SEQ ID NO: 136 - protein, 8 aa, glycine max
KFLVPPQE

SEQ ID NO: 137 - protein, 6 aa, glycine max
PQESQK

SEQ ID NO: 138 - protein, 6 aa, glycine max
CPSTYN

SEQ ID NO: 139 - protein, 6 aa, glycine max
QGGSQS

SEQ ID NO: 140 - protein, 5 aa, glycine max
SGAIV

SEQ ID NO: 141 - protein, 6 aa, glycine max
GGLRVT

SEQ ID NO: 142 - protein, 5 aa, glycine max
RLRQN

SEQ ID NO: 143 - protein, 6 aa, glycine max
LKLSAQ

SEQ ID NO: 144 - protein, 6 aa, glycine max
VAAKSQ

SEQ ID NO: 145 - protein, 6 aa, glycine max
PSIGNL

SEQ ID NO: 146 - protein, 7 aa, glycine max
FSFLVPP

SEQ ID NO: 147 - protein, 7 aa, glycine max
REQAQQN

SEQ ID NO: 148 - protein, 5 aa, glycine max
QKLNA

SEQ ID NO: 149 - protein, 6 aa, glycine max
RIESEG

SEQ ID NO: 150 - protein, 6 aa, glycine max
TYQEPQ

SEQ ID NO: 151 - protein, 5 aa, glycine max
PQESQ

SEQ ID NO: 152 - protein, 6 aa, glycine max
ESQQRG

SEQ ID NO: 153 - protein, 5 aa, glycine max
GRSQR

SEQ ID NO: 154 - protein, 6 aa, glycine max
QDRHQK

SEQ ID NO: 155 - protein, 6 aa, glycine max
VHRFRE

SEQ ID NO: 156 - protein, 6 aa, glycine max
EEENEG

SEQ ID NO: 157 - protein, 6 aa, glycine max
QIVRNL

SEQ ID NO: 158 - protein, 6 aa, glycine max
RKPQQE

SEQ ID NO: 159 - protein, 6 aa, glycine max
EEEQPQ

SEQ ID NO: 160 - protein, 6 aa, glycine max
DIYNPQ

SEQ ID NO: 161 - protein, 5 aa, glycine max
ERVFD

SEQ ID NO: 162 - protein, 5 aa, glycine max
ELQEG

SEQ ID NO: 163 - protein, 8 aa, glycine max
FREQPQQN

SEQ ID NO: 164 - protein, 6 aa, glycine max
QPQKQQ

SEQ ID NO: 165 - protein, 6 aa, glycine max
RQIVRK

SEQ ID NO: 166 - protein, 6 aa, glycine max
RRQQAR

SEQ ID NO: 167 - protein, 6 aa, glycine max
ECQIQR

SEQ ID NO: 168 - protein, 7 aa, glycine max
IQQGSGI

SEQ ID NO: 169 - protein, 6 aa, glycine max
QQKGQS

SEQ ID NO: 170 - protein, 6 aa, glycine max
IYHFRE

SEQ ID NO: 171 - protein, 6 aa, glycine max
AVPTGF

SEQ ID NO: 172 - protein, 5 aa, glycine max
NSFQN

SEQ ID NO: 173 - protein, 5 aa, glycine max
PTEEQ

SEQ ID NO: 174 - protein, 6 aa, glycine max
QQQRPE

SEQ ID NO: 175 - protein, 6 aa, glycine max
PDCDEK

SEQ ID NO: 176 - protein, 6 aa, glycine max
DKHCQS

SEQ ID NO: 177 - protein, 6 aa, glycine max
CQSQSR

SEQ ID NO: 178 - protein, 6 aa, glycine max
QSQSRN

SEQ ID NO: 179 - protein, 6 aa, glycine max
HNIGQT

SEQ ID NO: 180 - protein, 5 aa, glycine max
QGKGA

SEQ ID NO: 181 - protein, 6 aa, glycine max
VLSGFS

SEQ ID NO: 182 - protein, 6 aa, glycine max
LNECQL

SEQ ID NO: 183 - protein, 6 aa, glycine max
PDHRVE

SEQ ID NO: 184 - protein, 6 aa, glycine max
RNGLHS

SEQ ID NO: 185 - protein, 6 aa, glycine max
YSPYPR

SEQ ID NO: 186 - protein, 6 aa, glycine max
VAIPGC

SEQ ID NO: 187 - protein, 6 aa, glycine max
ETFEEP

SEQ ID NO: 188 - protein, 6 aa, glycine max
QQLQDS

SEQ ID NO: 189 - protein, 6 aa, glycine max
IRHFNE

SEQ ID NO: 190 - protein, 7 aa, glycine max
DIEYPET

SEQ ID NO: 191 - protein, 6 aa, glycine max
KQGQHQ

SEQ ID NO: 192 - protein, 6 aa, glycine max
DIAEKL

SEQ ID NO: 193 - protein, 6 aa, glycine max
KLESPD

SEQ ID NO: 194 - protein, 6 aa, glycine max
SVISPK

SEQ ID NO: 195 - protein, 5 aa, glycine max
QQDED

SEQ ID NO: 196 - protein, 5 aa, glycine max
KREQD

SEQ ID NO: 197 - protein, 6 aa, glycine max
HPPRRP

SEQ ID NO: 198 - protein, 6 aa, glycine max
REQDED

SEQ ID NO: 199 - protein, 6 aa, glycine max
GQDEDE

SEQ ID NO: 200 - protein, 6 aa, glycine max
TQPRRP

SEQ ID NO: 201 - protein, 6 aa, glycine max
PRQEEP

SEQ ID NO: 202 - protein, 6 aa, glycine max
EEPRER

SEQ ID NO: 203 - protein, 6 aa, glycine max
NSQHPE

SEQ ID NO: 204 - protein, 6 aa, glycine max
HPELQC

SEQ ID NO: 205 - protein, 6 aa, glycine max
QCAGVT

SEQ ID NO: 206 - protein, 5 aa, glycine max
TVSKR

SEQ ID NO: 207 - protein, 6 aa, glycine max
KGAIGF

SEQ ID NO: 208 - protein, 5 aa, glycine max
GSRSQ

SEQ ID NO: 209 - protein, 5 aa, glycine max
VTVEG

SEQ ID NO: 210 - protein, 7 aa, glycine max
RTPSYPP

SEQ ID NO: 211 - protein, 7 aa, glycine max
VEENICT

SEQ ID NO: 212 - protein, 6 aa, glycine max
PSRADF

SEQ ID NO: 213 - protein, 5 aa, glycine max
NCQGN

SEQ ID NO: 214 - protein, 6 aa, glycine max
RRGQLL

SEQ ID NO: 215 - protein, 5 aa, glycine max
PAVAE

SEQ ID NO: 216 - protein, 6 aa, glycine max
IPSEVL

SEQ ID NO: 217 - protein, 5 aa, glycine max
NSYNL

SEQ ID NO: 218 - protein, 5 aa, glycine max
CQLNN

SEQ ID NO: 219 - protein, 6 aa, glycine max
RVESEG

SEQ ID NO: 220 - protein, 6 aa, glycine max
YLPYPQ

SEQ ID NO: 221 - protein, 6 aa, glycine max
SHLPSY

SEQ ID NO: 222 - protein, 6 aa, glycine max
IGFAFP

SEQ ID NO: 223 - protein, 6 aa, glycine max
FPGCPE

SEQ ID NO: 224 - protein, 6 aa, glycine max
VIPLGV

SEQ ID NO: 225 - protein, 6 aa, glycine max
DEPVVA

SEQ ID NO: 226 - protein, 5 aa, glycine max
RVFYL

SEQ ID NO: 227 - protein, 6 aa, glycine max
GNPDIE

SEQ ID NO: 228 - protein, 6 aa, glycine max
IEHPET

SEQ ID NO: 229 - protein, 5 aa, glycine max
QGQHR

SEQ ID NO: 230 - protein, 5 aa, glycine max
HRQQE

SEQ ID NO: 231 - protein, 6 aa, glycine max
QQEEEG

SEQ ID NO: 232 - protein, 5 aa, glycine max
DTAEK

SEQ ID NO: 233 - protein, 5 aa, glycine max
EGGLS

SEQ ID NO: 234 - protein, 6 aa, glycine max
KWQEQE

SEQ ID NO: 235 - protein, 6 aa, glycine max
QEQEDE

SEQ ID NO: 236 - protein, 6 aa, glycine max
DEEYGR

SEQ ID NO: 237 - protein, 6 aa, glycine max
GKHEDD

SEQ ID NO: 238 - protein, 6 aa, glycine max
EEEDQP

SEQ ID NO: 239 - protein, 6 aa, glycine max
PRPDHP

SEQ ID NO: 240 - protein, 6 aa, glycine max
HPPQRP

SEQ ID NO: 241 - protein, 6 aa, glycine max
QRPSRP

SEQ ID NO: 242 - protein, 6 aa, glycine max
PEQQEP

SEQ ID NO: 243 - protein, 6 aa, glycine max
QQEPRG

SEQ ID NO: 244 - protein, 5 aa, glycine max
LRRGQ

SEQ ID NO: 245 - protein, 6 aa, glycine max
GNAVFD

SEQ ID NO: 246 - protein, 6 aa, glycine max
THHNAV

SEQ ID NO: 247 - protein, 6 aa, glycine max
SNSYNL

SEQ ID NO: 248 - protein, 6 aa, glycine max
LGQSQV

SEQ ID NO: 249 - protein, 5 aa, glycine max
PLVNP

SEQ ID NO: 250 - protein, 5 aa, glycine max
QGNSG

SEQ ID NO: 251 - protein, 5 aa, glycine max
LGGSL

SEQ ID NO: 252 - protein, 6 aa, glycine max
DDCCAL

SEQ ID NO: 253 - protein, 6 aa, glycine max
NSCGRS

SEQ ID NO: 254 - protein, 5 aa, glycine max
GNERC

SEQ ID NO: 255 - protein, 6 aa, glycine max
RAAPTG

SEQ ID NO: 256 - protein, 6 aa, glycine max
LCVGIP

SEQ ID NO: 257 - protein, 5 aa, glycine max
TEWSV

SEQ ID NO: 258 - protein, 6 aa, glycine max
VITQVK

SEQ ID NO: 259 - protein, 6 aa, glycine max
KYQGGC

SEQ ID NO: 260 - protein, 5 aa, glycine max
KPEEI

SEQ ID NO: 261 - protein, 6 aa, glycine max
FPQFKP

SEQ ID NO: 262 - protein, 5 aa, glycine max
EPGSL

SEQ ID NO: 263 - protein, 7 aa, glycine max
QGEPGAV

SEQ ID NO: 264 - protein, 6 aa, glycine max
ERLGDY

SEQ ID NO: 265 - protein, 6 aa, glycine max
AYVDDH

SEQ ID NO: 266 - protein, 8 aa, glycine max
NHLTHAAI

SEQ ID NO: 267 - protein, 6 aa, glycine max
IIGQDG

SEQ ID NO: 268 - protein, 6 aa, glycine max
GSVWLQ

SEQ ID NO: 269 - protein, 5 aa, glycine max
TPGQC

SEQ ID NO: 270 - protein, 5 aa, glycine max
IYDEP

SEQ ID NO: 271 - protein, 6 aa, glycine max
CDIEGN

SEQ ID NO: 272 - protein, 6 aa, glycine max
GSVWAQ

SEQ ID NO: 273 - protein, 5 aa, glycine max
VVERP

SEQ ID NO: 274 - protein, 5 aa, glycine max
IDQGY

SEQ ID NO: 275 - protein, 5 aa, glycine max
SIDEA

SEQ ID NO: 276 - protein, 6 aa, glycine max
NLGYSY

SEQ ID NO: 277 - protein, 7 aa, glycine max
VENVEGN

SEQ ID NO: 278 - protein, 6 aa, glycine max
EDEINS

SEQ ID NO: 279 - protein, 6 aa, glycine max
NSPVAP

SEQ ID NO: 280 - protein, 6 aa, glycine max
PDTAEK

SEQ ID NO: 281 - protein, 5 aa, glycine max
EKITF

SEQ ID NO: 282 - protein, 7 aa, glycine max
GSAGKLT

SEQ ID NO: 283 - protein, 5 aa, glycine max
ETKGD

SEQ ID NO: 284 - protein, 5 aa, glycine max
IGASA

SEQ ID NO: 285 - protein, 9 aa, glycine max
ATVQEKAER

SEQ ID NO: 286 - protein, 6 aa, glycine max
ARDPVQ

SEQ ID NO: 287 - protein, 6 aa, glycine max
ELATQK

SEQ ID NO: 288 - protein, 5 aa, glycine max
QHNTA

SEQ ID NO: 289 - protein, 6 aa, glycine max
GHGHHT

SEQ ID NO: 290 - protein, 6 aa, glycine max
GEYGQP

SEQ ID NO: 291 - protein, 5 aa, glycine max
HQTSA

SEQ ID NO: 292 - protein, 9 aa, glycine max
HGTGQPTGH

SEQ ID NO: 293 - protein, 6 aa, glycine max
HVTEGV

SEQ ID NO: 294 - protein, 7 aa, glycine max
VGSHPIG

SEQ ID NO: 295 - protein, 9 aa, glycine max
LFCIAHTCS

SEQ ID NO: 296 - protein, 7 aa, glycine max
SASKWQH

SEQ ID NO: 297 - protein, 6 aa, glycine max
QQDSCR

SEQ ID NO: 298 - protein, 6 aa, glycine max
SCRKQL

SEQ ID NO: 299 - protein, 7 aa, glycine max
KQLQGVN

SEQ ID NO: 300 - protein, 7 aa, glycine max
NLTPCEK

SEQ ID NO: 301 - protein, 5 aa, glycine max
QGRGD

SEQ ID NO: 302 - protein, 7 aa, glycine max
EDEEEEG

SEQ ID NO: 303 - protein, 5 aa, glycine max
QKCCT

SEQ ID NO: 304 - protein, 6 aa, glycine max
TEMSEL

SEQ ID NO: 305 - protein, 6 aa, glycine max
CKALQK

SEQ ID NO: 306 - protein, 9 aa, glycine max
NQSEELEEK

SEQ ID NO: 307 - protein, 6 aa, glycine max
MCRFGP

SEQ ID NO: 308 - protein, 6 aa, glycine max
IQCDLS

SEQ ID NO: 309 - protein, 8 aa, glycine max
CASKWQQH

SEQ ID NO: 310 - protein, 6 aa, glycine max
QHQQES

SEQ ID NO: 311 - protein, 7 aa, glycine max
ESCREQL

SEQ ID NO: 312 - protein, 6 aa, glycine max
NPCEHI

SEQ ID NO: 313 - protein, 6 aa, glycine max
EKIQAG

SEQ ID NO: 314 - protein, 7 aa, glycine max
DGSDEDH

SEQ ID NO: 315 - protein, 5 aa, glycine max
EGKEE

SEQ ID NO: 316 - protein, 6 aa, glycine max
KEEEEE

SEQ ID NO: 317 - protein, 5 aa, glycine max
SEMSE

SEQ ID NO: 318 - protein, 7 aa, glycine max
SPICQCK

SEQ ID NO: 319 - protein, 10 aa, glycine max
NQSEQLEGKE

SEQ ID NO: 320 - protein, 6 aa, glycine max
AIRCRL

SEQ ID NO: 321 - protein, 5 aa, glycine max
IGCDL

SEQ ID NO: 322 - protein, 8 aa, glycine max
NGACSPFE

SEQ ID NO: 323 - protein, 7 aa, glycine max
PPCRSRD

SEQ ID NO: 324 - protein, 5 aa, glycine max
RDCRC

SEQ ID NO: 325 - protein, 7 aa, glycine max
RCVPIGL

SEQ ID NO: 326 - protein, 6 aa, glycine max
AGFCIH

SEQ ID NO: 327 - protein, 6 aa, glycine max
GLSSVA

SEQ ID NO: 328 - protein, 8 aa, glycine max
HPNLCQSD

SEQ ID NO: 329 - protein, 6 aa, glycine max
QSDDEC

SEQ ID NO: 330 - protein, 8 aa, glycine max
GNFCARYP

SEQ ID NO: 331 - protein, 6 aa, glycine max
PPCRSS

SEQ ID NO: 332 - protein, 5 aa, glycine max
VDEHP

SEQ ID NO: 333 - protein, 5 aa, glycine max
SDSEA

SEQ ID NO: 334 - protein, 9 aa, glycine max
EGTSSAKLT

SEQ ID NO: 335 - protein, 6 aa, glycine max
RCVPIA

SEQ ID NO: 336 - protein, 6 aa, glycine max
LAPIDT

SEQ ID NO: 337 - protein, 5 aa, glycine max
TKPQT

SEQ ID NO: 338 - protein, 6 aa, glycine max
KPQTHA

SEQ ID NO: 339 - protein, 7 aa, glycine max
DPPNPHI

SEQ ID NO: 340 - protein, 5 aa, glycine max
DTFRN

SEQ ID NO: 341 - protein, 6 aa, glycine max
NLPHAS

SEQ ID NO: 342 - protein, 6 aa, glycine max
EIHVEK

SEQ ID NO: 343 - protein, 6 aa, glycine max
KHRVPK

SEQ ID NO: 344 - protein, 6 aa, glycine max
DHAGKA

SEQ ID NO: 345 - protein, 6 aa, glycine max
KESQRE

SEQ ID NO: 346 - protein, 5 aa, glycine max
ANVVG

SEQ ID NO: 347 - protein, 6 aa, glycine max
VAEKGR

SEQ ID NO: 348 - protein, 5 aa, glycine max
GRETE

SEQ ID NO: 349 - protein, 6 aa, glycine max
AHVVEG

SEQ ID NO: 350 - protein, 5 aa, glycine max
EYTAK

SEQ ID NO: 351 - protein, 7 aa, glycine max
EAQRELE

SEQ ID NO: 352 - protein, 6 aa, glycine max
QPQEAE

SEQ ID NO: 353 - protein, 5 aa, glycine max
GESEG

SEQ ID NO: 354 - protein, 7 aa, glycine max
RAKHEEG

SEQ ID NO: 355 - protein, 15 aa, glycine max
VFASQVVVQTEGRVC

SEQ ID NO: 356 - protein, 15 aa, glycine max
VFASQVVVQTEGRVC

SEQ ID NO: 357 - protein, 15 aa, glycine max
VFASQVVVQTEGRVC

SEQ ID NO: 358 - protein, 15 aa, glycine max
QVVVQTEGRVCESQS

SEQ ID NO: 359 - protein, 15 aa, glycine max
QTEGRVCESQSHGFH

SEQ ID NO: 360 - protein, 16 aa, glycine max
RVCESQSHGFHGLCNR

SEQ ID NO: 361 - protein, 15 aa, glycine max
SQSHGFHGLCNRDHN

SEQ ID NO: 362 - protein, 16 aa, glycine max
GFHGLCNRDHNCALVC

SEQ ID NO: 363 - protein, 15 aa, glycine max
LCNRDHNCALVCRNE

SEQ ID NO: 364 - protein, 15 aa, glycine max
RCKRSRRCFCTRICG

SEQ ID NO: 365 - protein, 15 aa, glycine max
PKHNKCLQSCNSERD

SEQ ID NO: 366 - protein, 15 aa, glycine max
PKHNKCLQSCNSERD

SEQ ID NO: 367 - protein, 15 aa, glycine max
SCNSERDSYRNQACH

SEQ ID NO: 368 - protein, 16 aa, glycine max
SCNSERDSYRNQACHA

SEQ ID NO: 369 - protein, 15 aa, glycine max
ERDSYRNQACHARCN

SEQ ID NO: 370 - protein, 15 aa, glycine max
LKVEKEECEEGEIPR

SEQ ID NO: 371 - protein, 15 aa, glycine max
KEECEEGEIPRPRPR

SEQ ID NO: 372 - protein, 15 aa, glycine max
KEECEEGEIPRPRPR

SEQ ID NO: 373 - protein, 16 aa, glycine max
IPRPRPRPQHPEREPQ

SEQ ID NO: 374 - protein, 16 aa, glycine max
RPRPQHPEREPQQPGE

SEQ ID NO: 375 - protein, 15 aa, glycine max
QHPEREPQQPGEKEE

SEQ ID NO: 376 - protein, 15 aa, glycine max
QHPEREPQQPGEKEE

SEQ ID NO: 377 - protein, 15 aa, glycine max
QPGEKEEDEDEQPRP

SEQ ID NO: 378 - protein, 15 aa, glycine max
EDEQPRPIPFPRPQP

SEQ ID NO: 379 - protein, 16 aa, glycine max
PRPIPFPRPQPRQEEE

SEQ ID NO: 380 - protein, 15 aa, glycine max
PQPRQEEEHEQREEQ

SEQ ID NO: 381 - protein, 15 aa, glycine max
WPRKEEKRGEKGSEE

SEQ ID NO: 382 - protein, 15 aa, glycine max
RPPHQKEERNEEEDE

SEQ ID NO: 383 - protein, 16 aa, glycine max
QKEERNEEEDEDEEQQ

SEQ ID NO: 384 - protein, 15 aa, glycine max
RNEEEDEDEEQQRES

SEQ ID NO: 385 - protein, 15 aa, glycine max
EDEDEEQQRESEESE

SEQ ID NO: 386 - protein, 15 aa, glycine max
KFEEINKVLFSREEG

SEQ ID NO: 387 - protein, 15 aa, glycine max
INKVLFSREEGQQQG

SEQ ID NO: 388 - protein, 15 aa, glycine max
SEDKPFNLRSRDPIY

SEQ ID NO: 389 - protein, 16 aa, glycine max
LSIVDMNEGALLLPHF

SEQ ID NO: 390 - protein, 15 aa, glycine max
ELVGLKEQQQEQQQE

SEQ ID NO: 391 - protein, 15 aa, glycine max
ELVGLKEQQQEQQQE

SEQ ID NO: 392 - protein, 16 aa, glycine max
LKEQQQEQQQEEQPLE

SEQ ID NO: 393 - protein, 15 aa, glycine max
LLKNQRESYFVDAQP

SEQ ID NO: 394 - protein, 15 aa, glycine max
LLKNQRESYFVDAQP

SEQ ID NO: 395 - protein, 15 aa, glycine max
PFLFGSNRFETLFKN

SEQ ID NO: 396 - protein, 16 aa, glycine max
RFETLFKNQYGRIRVL

SEQ ID NO: 397 - protein, 16 aa, glycine max
YRILEFNSKPNTLLLP

SEQ ID NO: 398 - protein, 16 aa, glycine max
TTYYVVNPDNNENLRL

SEQ ID NO: 399 - protein, 15 aa, glycine max
LRLITLAIPVNKPGR

SEQ ID NO: 400 - protein, 16 aa, glycine max
IPVNKPGRFESFFLSS

SEQ ID NO: 401 - protein, 15 aa, glycine max
SRNILEASYDTKFEE

SEQ ID NO: 402 - protein, 17 aa, glycine max
LFSREEGQQQGEQRLQE

SEQ ID NO: 403 - protein, 15 aa, glycine max
GEQRLQESVIVEISK

SEQ ID NO: 404 - protein, 16 aa, glycine max
LSKRAKSSSRKTISSE

SEQ ID NO: 405 - protein, 16 aa, glycine max
FFEITPEKNPQLRDLD

SEQ ID NO: 406 - protein, 16 aa, glycine max
SEQDIFVIPAGYPVVV

SEQ ID NO: 407 - protein, 15 aa, glycine max
QVQELAFPGSAQAVE

SEQ ID NO: 408 - protein, 15 aa, glycine max
RCNLLKVEEEEECEE

SEQ ID NO: 409 - protein, 15 aa, glycine max
QIPRPRPQHPERERQ

SEQ ID NO: 410 - protein, 16 aa, glycine max
PRPQHPERERQQHGEK

SEQ ID NO: 411 - protein, 16 aa, glycine max
ERQQHGEKEEDEGEQP

SEQ ID NO: 412 - protein, 15 aa, glycine max
HGEKEEDEGEQPRPF

SEQ ID NO: 413 - protein, 15 aa, glycine max
GEQPRPFPFPRPRQP

SEQ ID NO: 414 - protein, 15 aa, glycine max
RPFPFPRPRQPHQEE

SEQ ID NO: 415 - protein, 15 aa, glycine max
QEEEHEQKEEHEWHR

SEQ ID NO: 416 - protein, 15 aa, glycine max
QEEEHEQKEEHEWHR

SEQ ID NO: 417 - protein, 15 aa, glycine max
GGKGSEEEQDEREHP

SEQ ID NO: 418 - protein, 16 aa, glycine max
HKQEKHQGKESEEEEE

SEQ ID NO: 419 - protein, 16 aa, glycine max
HKQEKHQGKESEEEEE

SEQ ID NO: 420 - protein, 15 aa, glycine max
HQGKESEEEEEDQDE

SEQ ID NO: 421 - protein, 15 aa, glycine max
ESEEEEEDQDEDEEQ

SEQ ID NO: 422 - protein, 15 aa, glycine max
KESQESEGSESQREP

SEQ ID NO: 423 - protein, 15 aa, glycine max
KESQESEGSESQREP

SEQ ID NO: 424 - protein, 15 aa, glycine max
NKRSQQLQNLRDYRI

SEQ ID NO: 425 - protein, 15 aa, glycine max
KPNTLLLPHHADADY

SEQ ID NO: 426 - protein, 15 aa, glycine max
FEEINKVLFGREEGQ

SEQ ID NO: 427 - protein, 15 aa, glycine max
AIVVLVINEGEANIE

SEQ ID NO: 428 - protein, 15 aa, glycine max
NIELVGIKEQQQRQQ

SEQ ID NO: 429 - protein, 15 aa, glycine max
QDIFVIPAGYPVMVN

SEQ ID NO: 430 - protein, 15 aa, glycine max
ESYFVDAQPQQKEEG

SEQ ID NO: 431 - protein, 15 aa, glycine max
ESYFVDAQPQQKEEG

SEQ ID NO: 432 - protein, 15 aa, glycine max
ESYFVDAQPQQKEEG

SEQ ID NO: 433 - protein, 15 aa, glycine max
PLLLLGVVFLASVSV

SEQ ID NO: 434 - protein, 15 aa, glycine max
HFNSKRFQTLFKNQY

SEQ ID NO: 435 - protein, 15 aa, glycine max
ITQRNPQLRDLDVFL

SEQ ID NO: 436 - protein, 15 aa, glycine max
HPRPHQPHQKEEEKH

SEQ ID NO: 437 - protein, 15 aa, glycine max
LVNNDDRDSYNLQSG

SEQ ID NO: 438 - protein, 15 aa, glycine max
QSGDALRVPAGTTFY

SEQ ID NO: 439 - protein, 15 aa, glycine max
QRFNKRSPQLENLRD

SEQ ID NO: 440 - protein, 15 aa, glycine max
RDSYNLHPGDAQRIP

SEQ ID NO: 441 - protein, 15 aa, glycine max
RDSYNLHPGDAQRIP

SEQ ID NO: 442 - protein, 15 aa, glycine max
SHNILETSFHSEFEE

SEQ ID NO: 443 - protein, 15 aa, glycine max
SHNILETSFHSEFEE

SEQ ID NO: 444 - protein, 15 aa, glycine max
LETSFHSEFEEINRV

SEQ ID NO: 445 - protein, 15 aa, glycine max
NRVLFGEEEEQRQQE

SEQ ID NO: 446 - protein, 15 aa, glycine max
NRVLFGEEEEQRQQE

SEQ ID NO: 447 - protein, 15 aa, glycine max
FGEEEEQRQQEGVIV

SEQ ID NO: 448 - protein, 15 aa, glycine max
AIVILVINEGDANIE

SEQ ID NO: 449 - protein, 15 aa, glycine max
LLGTVFLASVCVSLK

SEQ ID NO: 450 - protein, 16 aa, glycine max
FLLFVLSGRAILTLVN

SEQ ID NO: 451 - protein, 15 aa, glycine max
VNPHDHQNLKIIKLA

SEQ ID NO: 452 - protein, 16 aa, glycine max
AIPVNKPGRYDDFFLS

SEQ ID NO: 453 - protein, 16 aa, glycine max
EGVIVELSKEQIRQLS

SEQ ID NO: 454 - protein, 16 aa, glycine max
GALLLPHFNSKAIVIL

SEQ ID NO: 455 - protein, 16 aa, glycine max
QKQKQEEEPLEVQRYR

SEQ ID NO: 456 - protein, 16 aa, glycine max
NVVRQIERQVQELAFP

SEQ ID NO: 457 - protein, 15 aa, glycine max
FSGCCFAFSSREQPQ

SEQ ID NO: 458 - protein, 15 aa, glycine max
FSGCCFAFSSREQPQ

SEQ ID NO: 459 - protein, 15 aa, glycine max
CFAFSSREQPQQNEC

SEQ ID NO: 460 - protein, 15 aa, glycine max
RPSYTNGPQEIYIQQ

SEQ ID NO: 461 - protein, 15 aa, glycine max
RPSYTNGPQEIYIQQ

SEQ ID NO: 462 - protein, 15 aa, glycine max
GMIYPGCPSTFEEPQ

SEQ ID NO: 463 - protein, 15 aa, glycine max
GMIYPGCPSTFEEPQ

SEQ ID NO: 464 - protein, 15 aa, glycine max
STFEEPQQPQQRGQS

SEQ ID NO: 465 - protein, 15 aa, glycine max
STFEEPQQPQQRGQS

SEQ ID NO: 466 - protein, 15 aa, glycine max
EPQQPQQRGQSSRPQ

SEQ ID NO: 467 - protein, 15 aa, glycine max
PQQRGQSSRPQDRHQ

SEQ ID NO: 468 - protein, 15 aa, glycine max
RPQDRHQKIYNFREG

SEQ ID NO: 469 - protein, 15 aa, glycine max
REGDLIAVPTGVAWW

SEQ ID NO: 470 - protein, 15 aa, glycine max
REGDLIAVPTGVAWW

SEQ ID NO: 471 - protein, 15 aa, glycine max
NSLENQLDQMPRRFY

SEQ ID NO: 472 - protein, 15 aa, glycine max
AGNQEQEFLKYQQEQ

SEQ ID NO: 473 - protein, 15 aa, glycine max
AGNQEQEFLKYQQEQ

SEQ ID NO: 474 - protein, 15 aa, glycine max
GHQSQKGKHQQEEEN

SEQ ID NO: 475 - protein, 15 aa, glycine max
GHQSQKGKHQQEEEN

SEQ ID NO: 476 - protein, 15 aa, glycine max
KQIAKNLQGENEGED

SEQ ID NO: 477 - protein, 15 aa, glycine max
GLSVIKPPTDEQQQR

SEQ ID NO: 478 - protein, 15 aa, glycine max
IKPPTDEQQQRPQEE

SEQ ID NO: 479 - protein, 15 aa, glycine max
DEKPQCKGKDKHCQR

SEQ ID NO: 480 - protein, 15 aa, glycine max
DEKPQCKGKDKHCQR

SEQ ID NO: 481 - protein, 15 aa, glycine max
FGSLRKNAMFVPHYN

SEQ ID NO: 482 - protein, 15 aa, glycine max
NNPFKFLVPPQESQK

SEQ ID NO: 483 - protein, 15 aa, glycine max
NNPFKFLVPPQESQK

SEQ ID NO: 484 - protein, 16 aa, glycine max
NNKPFQCAGVALSRCT

SEQ ID NO: 485 - protein, 16 aa, glycine max
KHCQRPRGSQSKSRRN

SEQ ID NO: 486 - protein, 18 aa, glycine max
KNAMFVPHYNLNANSIIY

SEQ ID NO: 487 - protein, 16 aa, glycine max
LNGRALIQVVNCNGER

SEQ ID NO: 488 - protein, 15 aa, glycine max
QNFVVAARSQSDNFE

SEQ ID NO: 489 - protein, 15 aa, glycine max
ARSQSDNFEYVSFKT

SEQ ID NO: 490 - protein, 15 aa, glycine max
FALREQAQQNECQIQ

SEQ ID NO: 491 - protein, 15 aa, glycine max
QIQKLNALKPDNRIE

SEQ ID NO: 492 - protein, 15 aa, glycine max
LNALKPDNRIESEGG

SEQ ID NO: 493 - protein, 15 aa, glycine max
FPGCPSTYQEPQESQ

SEQ ID NO: 494 - protein, 15 aa, glycine max
FPGCPSTYQEPQESQ

SEQ ID NO: 495 - protein, 15 aa, glycine max
ESQQRGRSQRPQDRH

SEQ ID NO: 496 - protein, 15 aa, glycine max
ESQQRGRSQRPQDRH

SEQ ID NO: 497 - protein, 15 aa, glycine max
QRPQDRHQKVHRFRE

SEQ ID NO: 498 - protein, 15 aa, glycine max
QRPQDRHQKVHRFRE

SEQ ID NO: 499 - protein, 15 aa, glycine max
QSQKGKQQEEENEGS

SEQ ID NO: 500 - protein, 15 aa, glycine max
AFGVNMQIVRNLQGE

SEQ ID NO: 501 - protein, 15 aa, glycine max
VTAPAMRKPQQEEDD

SEQ ID NO: 502 - protein, 15 aa, glycine max
PQQEEDDDDEEEQPQ

SEQ ID NO: 503 - protein, 15 aa, glycine max
QNIGQNSSPDIYNPQ

SEQ ID NO: 504 - protein, 15 aa, glycine max
NCNGERVFDGELQEG

SEQ ID NO: 505 - protein, 15 aa, glycine max
NCNGERVFDGELQEG

SEQ ID NO: 506 - protein, 16 aa, glycine max
MIFPGCPSTYQEPQES

SEQ ID NO: 507 - protein, 16 aa, glycine max
YQQQQQGGSQSQKGKQ

SEQ ID NO: 508 - protein, 15 aa, glycine max
NEEEDSGAIVTVKGG

SEQ ID NO: 509 - protein, 16 aa, glycine max
IVTVKGGLRVTAPAMR

SEQ ID NO: 510 - protein, 15 aa, glycine max
TICTMRLRQNIGQNS

SEQ ID NO: 511 - protein, 16 aa, glycine max
PALWLLKLSAQYGSLR

SEQ ID NO: 512 - protein, 16 aa, glycine max
PQNFAVAAKSQSDNFE

SEQ ID NO: 513 - protein, 16 aa, glycine max
KTNDRPSIGNLAGANS

SEQ ID NO: 514 - protein, 17 aa, glycine max
KNNNPFSFLVPPQESQR

SEQ ID NO: 515 - protein, 15 aa, glycine max
SFREQPQQNECQIQR

SEQ ID NO: 516 - protein, 15 aa, glycine max
IQQGSGIFGMIFPGC

SEQ ID NO: 517 - protein, 15 aa, glycine max
STFEEPQQKGQSSRP

SEQ ID NO: 518 - protein, 15 aa, glycine max
SRPQDRHQKIYHFRE

SEQ ID NO: 519 - protein, 15 aa, glycine max
FREGDLIAVPTGFAY

SEQ ID NO: 520 - protein, 15 aa, glycine max
TNSFQNQLDQMPRRF

SEQ ID NO: 521 - protein, 15 aa, glycine max
LSVISPPTEEQQQRP

SEQ ID NO: 522 - protein, 15 aa, glycine max
EEQQQRPEEEEKPDC

SEQ ID NO: 523 - protein, 15 aa, glycine max
PDCDEKDKHCQSQSR

SEQ ID NO: 524 - protein, 15 aa, glycine max
PDCDEKDKHCQSQSR

SEQ ID NO: 525 - protein, 15 aa, glycine max
PDCDEKDKHCQSQSR

SEQ ID NO: 526 - protein, 15 aa, glycine max
HCQSQSRNGIDETIC

SEQ ID NO: 527 - protein, 15 aa, glycine max
HNIGQTSSPDIFNPQ

SEQ ID NO: 528 - protein, 18 aa, glycine max
CFAFSFREQPQQNECQIQ

SEQ ID NO: 529 - protein, 16 aa, glycine max
EFLQYQPQKQQGGTQS

SEQ ID NO: 530 - protein, 17 aa, glycine max
AFVVDRQIVRKLQGENE

SEQ ID NO: 531 - protein, 16 aa, glycine max
QTFNLRRQQARQVKNN

SEQ ID NO: 532 - protein, 15 aa, glycine max
QLNNLNALEPDHRVE

SEQ ID NO: 533 - protein, 15 aa, glycine max
RNGLHSPSYSPYPRM

SEQ ID NO: 534 - protein, 15 aa, glycine max
RNGLHSPSYSPYPRM

SEQ ID NO: 535 - protein, 15 aa, glycine max
LGVAIPGCPETFEEP

SEQ ID NO: 536 - protein, 15 aa, glycine max
LGVAIPGCPETFEEP

SEQ ID NO: 537 - protein, 15 aa, glycine max
QQLQDSHQKIRHFNE

SEQ ID NO: 538 - protein, 15 aa, glycine max
QQLQDSHQKIRHFNE

SEQ ID NO: 539 - protein, 15 aa, glycine max
PDIEYPETMQQQQQQ

SEQ ID NO: 540 - protein, 15 aa, glycine max
QQQKSHGGRKQGQHQ

SEQ ID NO: 541 - protein, 15 aa, glycine max
NEDIAEKLESPDDER

SEQ ID NO: 542 - protein, 15 aa, glycine max
NEDIAEKLESPDDER

SEQ ID NO: 543 - protein, 15 aa, glycine max
LSVISPKWQEQQDED

SEQ ID NO: 544 - protein, 15 aa, glycine max
LSVISPKWQEQQDED

SEQ ID NO: 545 - protein, 15 aa, glycine max
SHPPRRPSHGKREQD

SEQ ID NO: 546 - protein, 15 aa, glycine max
SHPPRRPSHGKREQD

SEQ ID NO: 547 - protein, 15 aa, glycine max
RRPSHGKREQDEDED

SEQ ID NO: 548 - protein, 15 aa, glycine max
GKRNKTGQDEDEDED

SEQ ID NO: 549 - protein, 15 aa, glycine max
WRSKKTQPRRPRQEE

SEQ ID NO: 550 - protein, 15 aa, glycine max
KTQPRRPRQEEPRER

SEQ ID NO: 551 - protein, 15 aa, glycine max
KTQPRRPRQEEPRER

SEQ ID NO: 552 - protein, 15 aa, glycine max
HNAVTSYLKDVFRAI

SEQ ID NO: 553 - protein, 15 aa, glycine max
MIIIAQGKGALGVAI

SEQ ID NO: 554 - protein, 16 aa, glycine max
EEGGSVLSGFSKHFLA

SEQ ID NO: 555 - protein, 15 aa, glycine max
SSSKLNECQLNNLNA

SEQ ID NO: 556 - protein, 15 aa, glycine max
CQLNNLNALEPDHRV

SEQ ID NO: 557 - protein, 15 aa, glycine max
NLNALEPDHRVESEG

SEQ ID NO: 558 - protein, 15 aa, glycine max
NRNGSHLPSYLPYPQ

SEQ ID NO: 559 - protein, 15 aa, glycine max
NRNGSHLPSYLPYPQ

SEQ ID NO: 560 - protein, 15 aa, glycine max
AIGFAFPGCPETFEK

SEQ ID NO: 561 - protein, 15 aa, glycine max
AIGFAFPGCPETFEK

SEQ ID NO: 562 - protein, 15 aa, glycine max
FNEGDVLVIPLGVPY

SEQ ID NO: 563 - protein, 15 aa, glycine max
TYNTGDEPVVAISPL

SEQ ID NO: 564 - protein, 15 aa, glycine max
RVFYLAGNPDIEHPE

SEQ ID NO: 565 - protein, 15 aa, glycine max
RVFYLAGNPDIEHPE

SEQ ID NO: 566 - protein, 16 aa, glycine max
RVFYLAGNPDIEHPET

SEQ ID NO: 567 - protein, 15 aa, glycine max
QQQKSHGGRKQGQHR

SEQ ID NO: 568 - protein, 15 aa, glycine max
SHGGRKQGQHRQQEE

SEQ ID NO: 569 - protein, 15 aa, glycine max
RKQGQHRQQEEEGGS

SEQ ID NO: 570 - protein, 15 aa, glycine max
EDTAEKLRSPDDERK

SEQ ID NO: 571 - protein, 15 aa, glycine max
EGGLSVISPKWQEQE

SEQ ID NO: 572 - protein, 15 aa, glycine max
EGGLSVISPKWQEQE

SEQ ID NO: 573 - protein, 15 aa, glycine max
SVISPKWQEQEDEDE

SEQ ID NO: 574 - protein, 15 aa, glycine max
EQEDEDEDEDEEYGR

SEQ ID NO: 575 - protein, 15 aa, glycine max
SHGKHEDDEDEDEEE

SEQ ID NO: 576 - protein, 15 aa, glycine max
HEDDEDEDEEEDQPR

SEQ ID NO: 577 - protein, 15 aa, glycine max
EDEDEEEDQPRPDHP

SEQ ID NO: 578 - protein, 15 aa, glycine max
EEEDQPRPDHPPQRP

SEQ ID NO: 579 - protein, 15 aa, glycine max
QPRPDHPPQRPSRPE

SEQ ID NO: 580 - protein, 15 aa, glycine max
DHPPQRPSRPEQQEP

SEQ ID NO: 581 - protein, 15 aa, glycine max
RPEQQEPRGRGCQTR

SEQ ID NO: 582 - protein, 15 aa, glycine max
CQGNAVFDGELRRGQ

SEQ ID NO: 583 - protein, 15 aa, glycine max
CQGNAVFDGELRRGQ

SEQ ID NO: 584 - protein, 15 aa, glycine max
THHNAVSSYIKDVFR

SEQ ID NO: 585 - protein, 15 aa, glycine max
VLSNSYNLGQSQVRQ

SEQ ID NO: 586 - protein, 15 aa, glycine max
VLSNSYNLGQSQVRQ

SEQ ID NO: 587 - protein, 15 aa, glycine max
KYQGNSGPLVNPGSG

SEQ ID NO: 588 - protein, 15 aa, glycine max
KYQGNSGPLVNPGSG

SEQ ID NO: 589 - protein, 16 aa, glycine max
LIETWNSQHPELQCAG

SEQ ID NO: 590 - protein, 16 aa, glycine max
TWNSQHPELQCAGVTV

SEQ ID NO: 591 - protein, 16 aa, glycine max
QHPELQCAGVTVSKRT

SEQ ID NO: 592 - protein, 15 aa, glycine max
QCAGVTVSKRTLNRN

SEQ ID NO: 593 - protein, 16 aa, glycine max
IVVQGKGAIGFAFPGC

SEQ ID NO: 594 - protein, 15 aa, glycine max
QSSRRGSRSQQQLQD

SEQ ID NO: 595 - protein, 15 aa, glycine max
ERKQIVTVEGGLSVI

SEQ ID NO: 596 - protein, 17 aa, glycine max
DEEYGRTPSYPPRRPSH

SEQ ID NO: 597 - protein, 17 aa, glycine max
QTRNGVEENICTMKLHE

SEQ ID NO: 598 - protein, 16 aa, glycine max
ENIARPSRADFYNPKA

SEQ ID NO: 599 - protein, 15 aa, glycine max
RVRVVNCQGNAVFDG

SEQ ID NO: 600 - protein, 16 aa, glycine max
FDGELRRGQLLVVPQN

SEQ ID NO: 601 - protein, 15 aa, glycine max
VVPQNPAVAEQGGEQ

SEQ ID NO: 602 - protein, 16 aa, glycine max
DVFRVIPSEVLSNSYN

SEQ ID NO: 603 - protein, 15 aa, glycine max
SEVLSNSYNLGQSQV

SEQ ID NO: 604 - protein, 15 aa, glycine max
AYVDDHLLCGIEGNH

SEQ ID NO: 605 - protein, 15 aa, glycine max
GNHLTHAAIIGQDGS

SEQ ID NO: 606 - protein, 15 aa, glycine max
GNHLTHAAIIGQDGS

SEQ ID NO: 607 - protein, 15 aa, glycine max
THAAIIGQDGSVWLQ

SEQ ID NO: 608 - protein, 15 aa, glycine max
LIIGIYDEPMTPGQC

SEQ ID NO: 609 - protein, 15 aa, glycine max
DFPQFKPEEITAIMN

SEQ ID NO: 610 - protein, 16 aa, glycine max
LQSTDFPQFKPEEITA

SEQ ID NO: 611 - protein, 15 aa, glycine max
MNDFNEPGSLAPTGL

SEQ ID NO: 612 - protein, 17 aa, glycine max
KYMVIQGEPGAVIRGKK

SEQ ID NO: 613 - protein, 16 aa, glycine max
CNMVVERLGDYLIDQG

SEQ ID NO: 614 - protein, 15 aa, glycine max
MVVERPGDYLIDQGY

SEQ ID NO: 615 - protein, 15 aa, glycine max
MVVERPGDYLIDQGY

SEQ ID NO: 616 - protein, 16 aa, glycine max
DDHLLCDIEGNHLTHA

SEQ ID NO: 617 - protein, 16 aa, glycine max
IIGQDGSVWAQSTDFP

SEQ ID NO: 618 - protein, 15 aa, glycine max
HKIESIDEANLGYSY

SEQ ID NO: 619 - protein, 15 aa, glycine max
HKIESIDEANLGYSY

SEQ ID NO: 620 - protein, 15 aa, glycine max
VENVEGNGGPGTIKK

SEQ ID NO: 621 - protein, 16 aa, glycine max
GVFTFEDEINSPVAPA

SEQ ID NO: 622 - protein, 16 aa, glycine max
FEDEINSPVAPATLYK

SEQ ID NO: 623 - protein, 16 aa, glycine max
GGAALPDTAEKITFDS

SEQ ID NO: 624 - protein, 15 aa, glycine max
LPDTAEKITFDSKLV

SEQ ID NO: 625 - protein, 17 aa, glycine max
AGPNGGSAGKLTVKYET

SEQ ID NO: 626 - protein, 15 aa, glycine max
LTVKYETKGDAEPNQ

SEQ ID NO: 627 - protein, 19 aa, glycine max
LLISLLFCIAHTCSASKWQ

SEQ ID NO: 628 - protein, 17 aa, glycine max
IAHTCSASKWQHQQDSC

SEQ ID NO: 629 - protein, 16 aa, glycine max
SKWQHQQDSCRKQLQG

SEQ ID NO: 630 - protein, 16 aa, glycine max
QHQQDSCRKQLQGVNL

SEQ ID NO: 631 - protein, 17 aa, glycine max
QDSCRKQLQGVNLTPCE

SEQ ID NO: 632 - protein, 17 aa, glycine max
QLQGVNLTPCEKHIMEK

SEQ ID NO: 633 - protein, 15 aa, glycine max
IMEKIQGRGDDDDDD

SEQ ID NO: 634 - protein, 17 aa, glycine max
NEGKDEDEEEEGHMQKC

SEQ ID NO: 635 - protein, 15 aa, glycine max
EEGHMQKCCTEMSEL

SEQ ID NO: 636 - protein, 16 aa, glycine max
MQKCCTEMSELRSPKC

SEQ ID NO: 637 - protein, 16 aa, glycine max
SPKCQCKALQKIMENQ

SEQ ID NO: 638 - protein, 18 aa, glycine max
QKIMENQSEELEEKQKKK

SEQ ID NO: 639 - protein, 17 aa, glycine max
INLATMCRFGPMIQCDL

SEQ ID NO: 640 - protein, 14 aa, glycine max
RFGPMIQCDLSSDD

SEQ ID NO: 641 - protein, 17 aa, glycine max
IAHTCCASKWQQHQQES

SEQ ID NO: 642 - protein, 17 aa, glycine max
CCASKWQQHQQESCREQ

SEQ ID NO: 643 - protein, 17 aa, glycine max
QQHQQESCREQLKGINL

SEQ ID NO: 644 - protein, 16 aa, glycine max
KGINLNPCEHIMEKIQ

SEQ ID NO: 645 - protein, 16 aa, glycine max
CEHIMEKIQAGRRGED

SEQ ID NO: 646 - protein, 17 aa, glycine max
GRRGEDGSDEDHILIRT

SEQ ID NO: 647 - protein, 15 aa, glycine max
YIRKKEGKEEEEEGH

SEQ ID NO: 648 - protein, 16 aa, glycine max
RKKEGKEEEEEGHMQK

SEQ ID NO: 649 - protein, 15 aa, glycine max
MQKCCSEMSELKSPI

SEQ ID NO: 650 - protein, 17 aa, glycine max
MSELKSPICQCKALQKI

SEQ ID NO: 651 - protein, 18 aa, glycine max
QKIMDNQSEQLEGKEKKQ

SEQ ID NO: 652 - protein, 16 aa, glycine max
ELMNLAIRCRLGPMIG

SEQ ID NO: 653 - protein, 14 aa, glycine max
RLGPMIGCDLSSDD

SEQ ID NO: 654 - protein, 15 aa, glycine max
ETATNIGASAKAGME

SEQ ID NO: 655 - protein, 18 aa, glycine max
MEKTKATVQEKAERMTAR

SEQ ID NO: 656 - protein, 16 aa, glycine max
AERMTARDPVQKELAT

SEQ ID NO: 657 - protein, 16 aa, glycine max
DPVQKELATQKKEAKM

SEQ ID NO: 658 - protein, 15 aa, glycine max
KQAARQHNTAAKQSA

SEQ ID NO: 659 - protein, 16 aa, glycine max
TAGHMGHGHHTTGTGT

SEQ ID NO: 660 - protein, 16 aa, glycine max
TYSTTGEYGQPMGAHQ

SEQ ID NO: 661 - protein, 15 aa, glycine max
QPMGAHQTSAMPGHG

SEQ ID NO: 662 - protein, 17 aa, glycine max
SAMPGHGTGQPTGHVTE

SEQ ID NO: 663 - protein, 16 aa, glycine max
GQPTGHVTEGVVGSHP

SEQ ID NO: 664 - protein, 17 aa, glycine max
VTEGVVGSHPIGTNRGP

SEQ ID NO: 665 - protein, 18 aa, glycine max
EAADCNGACSPFEMPPCR

SEQ ID NO: 666 - protein, 17 aa, glycine max
SPFEMPPCRSRDCRCVP

SEQ ID NO: 667 - protein, 15 aa, glycine max
PPCRSRDCRCVPIGL

SEQ ID NO: 668 - protein, 17 aa, glycine max
RSRDCRCVPIGLVAGFC

SEQ ID NO: 669 - protein, 16 aa, glycine max
PIGLVAGFCIHPTGLS

SEQ ID NO: 670 - protein, 16 aa, glycine max
CIHPTGLSSVAKMIDE

SEQ ID NO: 671 - protein, 18 aa, glycine max
KMIDEHPNLCQSDDECMK

SEQ ID NO: 672 - protein, 16 aa, glycine max
HPNLCQSDDECMKKGS

SEQ ID NO: 673 - protein, 18 aa, glycine max
MKKGSGNFCARYPNNYID

SEQ ID NO: 674 - protein, 16 aa, glycine max
SPFEVPPCRSSDCRCV

SEQ ID NO: 675 - protein, 15 aa, glycine max
SVAKMVDEHPNLCQS

SEQ ID NO: 676 - protein, 15 aa, glycine max
GWCFDSDSEALKGFL

SEQ ID NO: 677 - protein, 16 aa, glycine max
MQEGTSSAKLTTHLNK

SEQ ID NO: 678 - protein, 16 aa, glycine max
RSSDCRCVPIALFVGF

SEQ ID NO: 679 - protein, 15 aa, glycine max
EIHVEKHRVPKMATH

SEQ ID NO: 680 - protein, 15 aa, glycine max
EIHVEKHRVPKMATH

SEQ ID NO: 681 - protein, 15 aa, glycine max
KDHAGKAMGDIGGRG

SEQ ID NO: 682 - protein, 15 aa, glycine max
HAAANVVGNKESQRE

SEQ ID NO: 683 - protein, 15 aa, glycine max
HAAANVVGNKESQRE

SEQ ID NO: 684 - protein, 15 aa, glycine max
ESGGQVVAEKGRETE

SEQ ID NO: 685 - protein, 15 aa, glycine max
ESGGQVVAEKGRETE

SEQ ID NO: 686 - protein, 15 aa, glycine max
AAAHVVEGAAGYAGH

SEQ ID NO: 687 - protein, 15 aa, glycine max
EYTAKKKEEAQRELE

SEQ ID NO: 688 - protein, 15 aa, glycine max
EYTAKKKEEAQRELE

SEQ ID NO: 689 - protein, 15 aa, glycine max
QPQEAEERPSEGIGE

SEQ ID NO: 690 - protein, 15 aa, glycine max
NTMGGESEGGGGKEE

SEQ ID NO: 691 - protein, 15 aa, glycine max
VLETRVTGRAKHEEG

SEQ ID NO: 692 - protein, 15 aa, glycine max
GLAFFLAPIDTKPQT

SEQ ID NO: 693 - protein, 15 aa, glycine max
GLAFFLAPIDTKPQT

SEQ ID NO: 694 - protein, 15 aa, glycine max
FLAPIDTKPQTHAGY

SEQ ID NO: 695 - protein, 15 aa, glycine max
FDTFRNSWDPPNPHI

SEQ ID NO: 696 - protein, 15 aa, glycine max
FDTFRNSWDPPNPHI

SEQ ID NO: 697 - protein, 15 aa, glycine max
ASNLPHASSNIDPLD

SEQ ID NO: 698 - protein, 18 aa, glycine max
GGYVPCRQEEDEELHHKC

SEQ ID NO: 699 - protein, 15 aa, glycine max
GVITQVKYQGGCGRG

SEQ ID NO: 700 - protein, 15 aa, glycine max
AFGGIRAAPTGNERC

SEQ ID NO: 701 - protein, 15 aa, glycine max
AFGGIRAAPTGNERC

SEQ ID NO: 702 - protein, 15 aa, glycine max
AVIMLCVGIPTEWSV

SEQ ID NO: 703 - protein, 15 aa, glycine max
AVIMLCVGIPTEWSV

SEQ ID NO: 704 - protein, 15 aa, glycine max
ILGGSLGTVDDCCAL

SEQ ID NO: 705 - protein, 15 aa, glycine max
ILGGSLGTVDDCCAL

SEQ ID NO: 706 - protein, 17 aa, glycine max
GIEPCSKYSQHQQHVQN

SEQ ID NO: 707 - protein, 17 aa, glycine max
GEIMCWREVVTQHQQHA

SEQ ID NO: 708 - protein, 15 aa, glycine max
VFASQVVVQTEGRVC

SEQ ID NO: 709 - protein, 17 aa, glycine max
GGFASQVFADCCKLQSD

SEQ ID NO: 710 - protein, 17 aa, glycine max
GEFASQVEHIRPVLCAY

SEQ ID NO: 711 - protein, 17 aa, glycine max
GDFASQVNQLNWFVFPN

SEQ ID NO: 712 - protein, 17 aa, glycine max
GIFASQVRIIAIHYPNF

SEQ ID NO: 713 - protein, 17 aa, glycine max
GQFASQVLVEFCDQSNC

SEQ ID NO: 714 - protein, 17 aa, glycine max
GPFASQVLKKLCAQYAF

SEQ ID NO: 715 - protein, 17 aa, glycine max
GVFASQVSQGQQQDQFD

SEQ ID NO: 716 - protein, 17 aa, glycine max
GFFSCFASQVICYGGKD

SEQ ID NO: 717 - protein, 17 aa, glycine max
GWDHCEASQVVKFNYLC

SEQ ID NO: 718 - protein, 17 aa, glycine max
GIWASQVVWKFQTGPYF

SEQ ID NO: 719 - protein, 17 aa, glycine max
GHSASQVVHNVREGFSY

SEQ ID NO: 720 - protein, 17 aa, glycine max
GQYASQVVHSKEHPDTN

SEQ ID NO: 721 - protein, 17 aa, glycine max
GYNMSKYHPASQVVTYY

SEQ ID NO: 722 - protein, 17 aa, glycine max
GYLASQVVIYPESEEQF

SEQ ID NO: 723 - protein, 17 aa, glycine max
GNYASQVVQVFGTNDFN

SEQ ID NO: 724 - protein, 17 aa, glycine max
GVPASQVVDNKQTLVYF

SEQ ID NO: 725 - protein, 17 aa, glycine max
GLYASQVVSVRYYDYSC

SEQ ID NO: 726 - protein, 17 aa, glycine max
GQPASQVVIAQELFFEC

SEQ ID NO: 727 - protein, 17 aa, glycine max
GFNASQVVNKPECDVEY

SEQ ID NO: 728 - protein, 17 aa, glycine max
GHRASQVVWAYEYAHHA

SEQ ID NO: 729 - protein, 17 aa, glycine max
GDYASQVVQPQGEFQPC

SEQ ID NO: 730 - protein, 17 aa, glycine max
GQQASQVVIQKCDQPDY

SEQ ID NO: 731 - protein, 17 aa, glycine max
GVEASQVVAFGICKQVA

SEQ ID NO: 732 - protein, 17 aa, glycine max
GTEASQVVVSACHQCII

SEQ ID NO: 733 - protein, 17 aa, glycine max
GWDHCEASQVVKHVREI

SEQ ID NO: 734 - protein, 12 aa, Artificial Sequence
  peptide P1
GFNLCNRDRPAP

SEQ ID NO: 735 - protein, 17 aa, Artificial Sequence
  peptide P2
SQSHGFHGLCNRDHNCA

SEQ ID NO: 736 - protein, 11 aa, Artificial Sequence
  peptide P3
EVSFASQVLIY

SEQ ID NO: 737 - protein, 10 aa, Artificial Sequence
  peptide P4
SHHDQPRQHA

SEQ ID NO: 738 - protein, 11 aa, Artificial Sequence
  peptide P5
HITFSREEGSI

SEQ ID NO: 739 - protein, 12 aa, Artificial Sequence
  peptide P6
KVLFSREEGQQQ

SEQ ID NO: 740 - protein, 15 aa, Artificial Sequence
  peptide P7
DEGEQPRPFPFPRPR

SEQ ID NO: 741 - protein, 11 aa, Artificial Sequence
  peptide P8
HIQEEECEGDL

SEQ ID NO: 742 - protein, 12 aa, Artificial Sequence
  peptide P9
VEEEEECEEGQI

SEQ ID NO: 743 - protein, 11 aa, Artificial Sequence
  peptide P10
SDKYQEEFQPR

SEQ ID NO: 744 - protein, 10 aa, Artificial Sequence
  peptide P11
TEKYQQNSSG

SEQ ID NO: 745 - protein, 10 aa, Artificial Sequence
  peptide P12
EFLKYQQEQG

SEQ ID NO: 746 - protein, 17 aa, Artificial Sequence
  peptide P13
GVYNSQVDDEEEQNQRD

SEQ ID NO: 747 - protein, 9 aa, Artificial Sequence
  peptide P14
HVVEQEFLD

SEQ ID NO: 748 - protein, 14 aa, Artificial Sequence
  peptide P15
RRFYLAGNQEQEFL

SEQ ID NO: 749 - protein, 9 aa, Artificial Sequence
  peptide P16
ISQQDRHRI

SEQ ID NO: 750 - protein, 10 aa, Artificial Sequence
  peptide P17
FDQKESQHFS

SEQ ID NO: 751 - protein, 16 aa, Artificial Sequence
  peptide P18
HAANVVGNKESQREAR

SEQ ID NO: 752 - protein, 17 aa, Artificial Sequence
  peptide P19
GYNPCRQEEDEELHHKC

SEQ ID NO: 753 - protein, 10 aa, Artificial Sequence
  peptide P20
QDQEEDEEDE